(19) United States
(12) Patent Application Publication
(10) Pub. No.: US 2015/0278438 A1
(43) Pub. Date: Oct. 1, 2015

(54) GENETIC PREDICTORS OF RESPONSE TO TREATMENT WITH CRHR1 ANTAGONISTS

(71) Applicant: MAX-PLANCK-GESELLSCHAFT ZUR FÖRDERUNG DER WISSENSCHAFTEN E.V., München (DE)

(72) Inventors: Bertram Müller-Myhsok, Munchen (DE); Elisabeth Binder, Munchen (DE); Florian Holsboer, Munchen (DE)

(21) Appl. No.: 14/396,477

(22) PCT Filed: Apr. 23, 2013
(86) PCT No.: PCT/EP2013/058411

§ 371 Date: Oct. 23, 2014

(30) Foreign Application Priority Data
Apr. 23, 2012 (EP) 12165224.2
Apr. 23, 2012 (GB) 1207089.2

(51) Int. Cl. G06F 19/22; G06F 19/00; A61K 31/44; A61K 31/426; C12Q 1/68; A61K 31/53

(52) U.S. Cl. CPC G06F 19/22; C12Q 1/6883; A61K 31/53; A61K 31/44; A61K 31/426; G06F 19/345; C12Q 2600/156; C12Q 2600/106; C12Q 2600/16

(57) ABSTRACT

The invention relates inter alia to methods for predicting the response of patients with depressive symptoms and/or anxiety symptoms to treatment with a CRHR1 antagonist, and algorithms, kits, microarrays, probes and or/primers for use in such methods.

### FIELD OF THE INVENTION

The invention relates inter alia to methods for predicting the response of patients with depressive symptoms and/or anxiety symptoms to treatment with a CRHR1 antagonist, and algorithms, kits, microarrays, probes and or/primers for use in such methods.

### BACKGROUND OF THE INVENTION

While current antidepressant drugs can provide effective treatments of patients with depression and/or anxiety symptoms in a number of psychiatric disorders, some of the patients may only show partial remission of symptoms or do not respond at all (Trivedi et al., Am J Psychiatry, 2006). This may be due to the fact that these drugs do not target the inherent pathophysiologic disturbances leading to the clinical condition in these patients. A number of antidepressant strategies, derived from both animal studies and human studies have been tested, but so far with little success. One of these approaches is the use of corticotropin releasing hormone receptor type 1 (CRHR1) antagonists. Increased activity or concentrations of its ligand corticotropin releasing hormone (CRH) in the brain or the cerebrospinal fluid have been shown to be associated with depression and anxiety in humans (Nemeroff et al., Arch Gen Psychiatry, 1988; Nemeroff et al. Science, 1984; Purba et al. Arch Gen Psychiatry, 1996; Carpenter, et al. Neuropsychopharmacology, 2004), primates (Coplan et al., Proc Natl Acad Sci USA, 1996; Sanchez et al., Dev Psychopathol, 2001) and rodents (Muller et al. Nat Neurosci., 2003; Timpl et al., Nat Genet. 1998). In addition, molecular studies in experimental animals and open label studies in human patients indicate that CRHR1 may be suitable in the treatment of depression and anxiety (Ising et al., Exp Clin Psychopharmacol., 2007; Holsboer et al., CNS Spectr., 2001; Paez-Pereda et al., Expert Opin Investig Drugs, 2011). However, so far all randomized clinical trials have failed to demonstrate the superiority of this drug to placebo (Coric et al., Depress Anxiety, 2010; Binneman et al., Am J Psychiatry, 2008).

Hence, there is still uncertainty whether CRHR1 antagonists are indeed suitable to treat depressive symptoms and/or anxiety symptoms in patients in need thereof. In order to establish the chances of such a therapy it would be desirable to provide reliable methods for predicting a treatment response to CRHR1 antagonists in patients with depressive symptoms and/or anxiety symptoms.

### SUMMARY OF THE INVENTION

One aspect of the present inventions relates to a method for providing an algorithm for predicting a treatment response to CRHR1 antagonists in patients with depressive symptoms and/or anxiety symptoms. The method may comprise the following steps:

(a) performing a single nucleotide polymorphism (SNP) genotyping analysis in a group of patients with depressive symptoms and/or anxiety symptoms;

(b) determining a value indicative for CRH activity in each patient of the group, wherein a value indicative for CRH overactivity is indicative for a patient responding to a treatment with a CRH1 antagonist;

(c) identifying at least one SNP associated with a value indicative for CRH overactivity as determined in step (b); and

(d) determining the algorithm by machine-learning from the association of the at least one SNP identified in step (c) with the value indicative for CRH overactivity.

In one embodiment of the invention, the SNP genotyping analysis is performed in a group of at least 10 patients.

In another embodiment, the SNP genotyping analysis comprises the use of SNP-specific primers, SNP-specific probes, a primer extension reaction, the use of SNP microarrays and/or the use of sequencing methods.

Typically, the value indicative for CRH overactivity in each patient is determined by measuring ACTH response and/or cortisol response to a combined dexamethasone suppression/CRH stimulation test in each patient.

Usually, at least 20, optionally at least 25 or at least 30 SNPs associated with the value indicative for CRH overactivity are identified in step (c).

In one embodiment machine-learning is selected from the group consisting of artificial neural network learning, decision tree learning, support vector machine learning, Bayesian network learning, clustering and regression analysis.

In a specific embodiment, artificial neural network learning is used as the machine-learning method.

In one embodiment, in step (d), a number N of SNPs identified in step (c) is associated with a value indicative for CRH overactivity, wherein N is sufficient to provide an algorithm having an accuracy of prediction of at least 80% and/or a sensitivity of prediction of at least 70% and/or a specificity of prediction of at least 70% and/or a positive predictive value of prediction of at least 70% and/or a negative predictive value of prediction of at least 70%. N may be at least 20 or at least 25, such as 30.

In one embodiment, step (c) further comprises identifying at least one SNP associated with a value indicative for normal CRH activity as determined in step (b) of the above described method. Further, in this embodiment, step (d) may further comprise machine-learning from the association of the at least one SNP identified in step (c) with the value indicative for normal CRH overactivity.

In another embodiment, the algorithm determined in step (d) associates at least one SNP selected from the group consisting of SNPs described in table 1 below and an SNP in strong linkage disequilibrium with any of the foregoing SNPs with a value indicative for CRH overactivity or normal CRH activity.

In a further embodiment, the algorithm determined in step (d) associates at least 20, optionally at least 25 or at least 30 SNPs selected from the group consisting of SNPs described in table 1 and an SNP in strong linkage disequilibrium with any of the foregoing SNPs with a value indicative for CRH overactivity or normal CRH activity.

In a specific embodiment, the algorithm determined in step (d) associates all of the SNPs described in table 1 with CRH overactivity or normal CRH activity.

Another aspect of the invention is a method for predicting a treatment response to CRHR1 antagonists in patients with depressive symptoms and/or anxiety symptoms, wherein the method comprises the following steps:

(a) determining in a nucleic acid sample obtained from a patient the presence or absence of at least one single nucleotide polymorphism (SNP) associated with a value indicative for CRH overactivity;

(b) predicting the treatment response to CRHR1 antagonists by linking an algorithm provided by the method for providing an algorithm as described above with the presence or absence of the at least one SNP determined in step (a).

Step (a) may comprise determining at least one of the SNPs, optionally all of the SNPs which were associated with a value indicative for CRH overactivity when determining the algorithm by machine-learning from this association.

In one embodiment of the method for predicting a treatment response to CRHR1 antagonists in patients with depressive symptoms and/or anxiety symptoms, the method comprises a further step of determining a value indicative for the rapid-eye-movement (REM) density, e.g. during a first REM night sleep episode of a patient.

In one embodiment, the method for predicting a treatment response is preceded by a step of obtaining a nucleic acid sample from a patient.

The method for predicting a treatment response as described above may further comprise in step (a) determining at least one SNP associated with a value indicative for normal CRH activity.

In another embodiment, in step (a) the presence or absence of a number of SNPs is determined, wherein the SNPs optionally correspond to the SNPs which were sufficient to provide an algorithm having an accuracy of prediction of at least 80% and/or a sensitivity of prediction of at least 70% and/or a specificity of prediction of at least 70% and/or a positive predictive value of prediction of at least 70% and/or a negative predictive value of prediction of at least 70%. N may be at least 20 or at least 25, such as 30.

In a further embodiment at least 20, optionally at least 25 or at least 30 SNPs selected from the group of SNPs consisting of SNPs as described in table 1 below and an SNP in strong linkage disequilibrium with any of the foregoing SNPs are determined.

In a specific embodiment all of the SNPs described in table 1 are determined.

Typically, in the method for predicting a treatment response to CRHR1 antagonists the at least one SNP associated with a value indicative for CRH overactivity or for normal CRH activity is determined by using SNP-specific primers, SNP-specific probes, a primer extension reaction, SNP microarrays and/or sequencing methods. Another aspect of the invention concerns a group of biomarkers, comprising:
SNP rs6437726,
SNP rs1986684,
SNP rs7380830,
SNP rs3903768,
SNP rs7325978,
SNP rs13585,
SNP rs9368373,
SNP rs10935354,
SNP rs8095703,
SNP rs10206851,
SNP rs9542977,
SNP rs4942879,
SNP rs9542954,
SNP rs1593478,
SNP rs9542951,
SNP rs2188534,
SNP rs12524124,
SNP rs4352629,
SNP rs7448716,
SNP rs11873533,
SNP rs10062658,
SNP rs12547917,
SNP rs1038268,
SNP rs2375811,
SNP rs1352671,
SNP rs364331,
SNP rs1924949,
SNP rs11025990,
SNP rs3758562, and
SNP rs10156056.

In one embodiment, the group of biomarkers is a group of biomarkers, wherein
SNP rs6437726 is represented by a single polymorphic change at position 201 of SEQ ID NO: 1, wherein in one or two alleles the wild-type nucleotide A is replaced by indicator nucleotide G,
SNP rs1986684 is represented by a single polymorphic change at position 201 of SEQ ID NO: 2, wherein in one or two alleles the wild-type nucleotide A is replaced by indicator nucleotide G,
SNP rs7380830 is represented by a single polymorphic change at position 201 of SEQ ID NO: 3, wherein in one or two alleles the wild-type nucleotide C is replaced by indicator nucleotide T,
SNP rs3903768 is represented by a single polymorphic change at position 201 of SEQ ID NO: 4, wherein in one or two alleles the wild-type nucleotide A is replaced by indicator nucleotide G,
SNP rs7325978 is represented by a single polymorphic change at position 201 of SEQ ID NO: 5, wherein in one or two alleles the wild-type nucleotide C is replaced by indicator nucleotide T,
SNP rs13585 is represented by a single polymorphic change at position 185 of SEQ ID NO: 6, wherein in one or two alleles the wild-type nucleotide C is replaced by indicator nucleotide T,
SNP rs9368373 is represented by a single polymorphic change at position 201 of SEQ ID NO: 7, wherein in one or two alleles the wild-type nucleotide C is replaced by indicator nucleotide T,
SNP rs10935354 which is represented by a single polymorphic change at position 201 of SEQ ID NO: 8, wherein in one or two alleles the wild-type nucleotide A is replaced by indicator nucleotide G,
SNP rs8095703 is represented by a single polymorphic change at position 201 of SEQ ID NO: 9, wherein in one or two alleles the wild-type nucleotide A is replaced by indicator nucleotide G,
SNP rs10206851 is represented by a single polymorphic change at position 201 of SEQ ID NO: 10, wherein in one or two alleles the wild-type nucleotide C is replaced by indicator nucleotide T,
SNP rs9542977 is represented by a single polymorphic change at position 201 of SEQ ID NO: 11, wherein in one or two alleles the wild-type nucleotide C is replaced by indicator nucleotide T,
SNP rs4942879 is represented by a single polymorphic change at position 201 of SEQ ID NO: 12, wherein in one or two alleles the wild-type nucleotide A is replaced by indicator nucleotide G,
SNP rs9542954 is represented by a single polymorphic change at position 201 of SEQ ID NO: 13, wherein in one or two alleles the wild-type nucleotide A is replaced by indicator nucleotide C,
SNP rs1593478 is represented by a single polymorphic change at position 201 of SEQ ID NO: 14, wherein in one or two alleles the wild-type nucleotide C is replaced by indicator nucleotide T,
SNP rs9542951 is represented by a single polymorphic change at position 201 of SEQ ID NO: 15, wherein in one or two alleles the wild-type nucleotide A is replaced by indicator nucleotide G,
SNP rs2188534 is represented by a single polymorphic change at position 200 of SEQ ID NO: 16, wherein in one or two alleles the wild-type nucleotide G is replaced by indicator nucleotide T,
SNP rs12524124 is represented by a single polymorphic change at position 201 of SEQ ID NO: 17, wherein in one or two alleles the wild-type nucleotide A is replaced by indicator nucleotide G,
SNP rs4352629 is represented by a single polymorphic change at position 201 of SEQ ID NO: 18, wherein in one or two alleles the wild-type nucleotide C is replaced by indicator nucleotide T,
SNP rs7448716 is represented by a single polymorphic change at position 201 of SEQ ID NO: 19, wherein in one or two alleles the wild-type nucleotide A is replaced by indicator nucleotide G,
SNP rs11873533 is represented by a single polymorphic change at position 201 of SEQ ID NO: 20, wherein in one or two alleles the wild-type nucleotide A is replaced by indicator nucleotide C,
SNP rs10062658 is represented by a single polymorphic change at position 201 of SEQ ID NO: 21, wherein in one or two alleles the wild-type nucleotide A is replaced by indicator nucleotide G,
SNP rs12547917 is represented by a single polymorphic change at position 201 of SEQ ID NO: 22, wherein in one or two alleles the wild-type nucleotide C is replaced by indicator nucleotide T,
SNP rs1038268 is represented by a single polymorphic change at position 201 of SEQ ID NO: 23, wherein in one or two alleles the wild-type nucleotide C is replaced by indicator nucleotide T,
SNP rs2375811 is represented by a single polymorphic change at position 201 of SEQ ID NO: 24, wherein in one or two alleles the wild-type nucleotide A is replaced by indicator nucleotide G,
SNP rs1352671 is represented by a single polymorphic change at position 201 of SEQ ID NO: 25, wherein in one or two alleles the wild-type nucleotide A is replaced by indicator nucleotide C,
SNP rs364331 is represented by a single polymorphic change at position 201 of SEQ ID NO: 26, wherein in one or two alleles the wild-type nucleotide A is replaced by indicator nucleotide C,
SNP rs1924949 is represented by a single polymorphic change at position 201 of SEQ ID NO: 27, wherein in one or two alleles the wild-type nucleotide A is replaced by indicator nucleotide G,
SNP rs11025990 is represented by a single polymorphic change at position 201 of SEQ ID NO: 28, wherein in one or two alleles the wild-type nucleotide A is replaced by indicator nucleotide G,
SNP rs3758562 is represented by a single polymorphic change at position 201 of SEQ ID NO: 29, wherein in one or two alleles the wild-type nucleotide A is replaced by indicator nucleotide G, and
SNP rs10156056 is represented by a single polymorphic change at position 201 of SEQ ID NO: 30, wherein in one or two alleles the wild-type nucleotide C is replaced by indicator nucleotide G.

The group of biomarkers as described above may constitute a marker for a treatment response to CRHR1 antagonists in patients with depressive and/or anxiety symptoms.

Further, the group of biomarkers may additionally comprise REM density.

Another aspect of the invention concerns a method for detecting CRH overactivity in a patient with depressive symptoms and/or anxiety symptoms, comprising determining the status of a biomarker or a group of biomarkers as defined above in a nucleic acid isolated from a patient's sample, wherein the presence of indicator nucleotides as defined above is indicative for CRH overactivity.

Another aspect of the invention concerns a method for monitoring depression and/or anxiety therapy of a patient with a CRHR1 antagonist comprising the step of determining the status of a biomarker or a group of biomarkers as defined above before and during the therapy, optionally also after the therapy.

Another aspect of the invention concerns a method of identifying a patient with depressive symptoms and/or anxiety symptoms as eligible for a therapy with a CRHR1 antagonist, comprising:

(a) determining in a nucleic acid sample isolated from a patient's sample the status of a biomarker or a group of biomarkers as defined above;

(b) identifying the patient as eligible for a therapy with a CRHR1 antagonist, where the algorithm provided by the method of claim 1 predicts that patient responds to the treatment with CRHR1 antagonists.

Another aspect of the invention concerns a method of identifying a patient with depressive symptoms and/or anxiety symptoms as eligible for a therapy with a CRHR1 antagonist, comprising:

(a) determining in a nucleic acid sample isolated from a patient's sample the status of a biomarker or a group of biomarkers as defined above;

(b) identifying the patient as eligible for a therapy with a CRHR1 antagonist, where the patient's sample is classified as showing the presence of indicator nucleotides as defined above.

In some embodiments of the above methods of identifying a patient with depressive symptoms and/or anxiety symptoms as eligible for a therapy with a CRHR1 antagonist, the method may further comprise a step of administering a CRHR1 antagonist.

In some embodiments of the above methods of identifying a patient with depressive symptoms and/or anxiety symptoms as eligible for a therapy with a CRHR1 antagonist, the CRHR1 antagonist may be selected from the group consisting of CP154,526, Antalarmin, CRA 5626, Emicerfont, DMP-696, DMP-904, DMP-695, SC-241, BMS-561388, Pexacerfont, R121919, NBI30545, PD-171729, Verucerfont, NBI34041, NBI35965, SN003, CRA0450, SSR125543A, CP-316,311, CP-376,395, NBI-27914, ONO-2333Ms, NBI-34101, PF-572778, GSK561579 and GSK586529.

A further aspect of the invention is a composition for the analysis of at least one single nucleotide polymorphism (SNP) indicative for the treatment response to CRHR1 antagonists in patients with depressive symptoms and/or anxiety symptoms, comprising a nucleic acid affinity ligand for a biomarker as defined above.

A further aspect of the invention is a kit, diagnostic composition or device for the analysis of at least one single nucleotide polymorphism (SNP) indicative for the treatment response to CRHR1 antagonists in patients with depressive symptoms and/or anxiety symptoms, comprising at least one primer and/or probe selective for determining the presence or absence of at least one SNP associated with a value indicative for CRH overactivity.

Usually the kit, diagnostic composition or device further comprises an enzyme for primer elongation, nucleotides and/or labeling agents.

Another aspect of the invention concerns an microarray for the analysis of at least one single nucleotide polymorphism (SNP) indicative for the treatment response to CRHR1 antagonists in patients with depressive symptoms and/or anxiety symptoms, comprising at least one probe selective for determining the presence or absence of at least one SNP associated with the value indicative for CRH overactivity.

Another aspect of the invention concerns a primer or probe for the analysis of at least one single nucleotide polymorphism (SNP) associated with a value indicative for CRH overactivity.

A further aspect of the invention is the use of a microarray as described above for predicting a treatment response to CRHR1 antagonists in patients with depressive symptoms and/or anxiety symptoms.

A further aspect of the invention is the use of a group of biomarkers as defined in above for detecting CRH overactivity in patients with depressive symptoms and/or anxiety symptoms, or for screening a population of patients with depressive symptoms and/or anxiety symptoms for eligibility for a therapy with a CRHR1 antagonist.

Another aspect of the inventions deals with a computer program product designed such that a method for predicting a treatment response to CRHR1 antagonists in patients with depressive symptoms and/or anxiety symptoms is performed when the computer program product is used on a computer, wherein the method comprises linking the algorithm provided by the method for providing an algorithm as described above with the presence or absence of at least one single nucleotide polymorphism (SNP) associated with a value indicative for CRH overactivity in a nucleic acid sample of a patient.

A further aspect of the invention relates to a machine-readable medium with instructions which can be performed on a computer for the performance of a method for predicting a treatment response to CRHR1 antagonists in patients with depressive symptoms and/or anxiety symptoms, wherein the method comprises linking the algorithm provided by the method for providing an algorithm as described above with the presence or absence of at least one single nucleotide polymorphism (SNP) associated with a value indicative for CRH overactivity in a nucleic acid sample of a patient.

Further, an aspect of the invention refers to a method for providing an algorithm for predicting a treatment response to a compound for treating depressive symptoms and/or anxiety symptoms in patients who have CRH overactivity, wherein the method comprises the following steps:

(a) performing a single nucleotide polymorphism (SNP) genotyping analysis in a group of patients with depressive symptoms and/or anxiety symptoms;

(b) determining a value indicative for CRH activity in each patient of the group;

(c) identifying at least one SNP associated with a value indicative for CRH overactivity as determined in step (b);

(d) determining the algorithm by machine-learning from the association of the at least one SNP identified in step (c) with the value indicative for CRH overactivity.

An additional aspect of the invention refers to a method for predicting a treatment response to a compound for treating depressive symptoms and/or anxiety symptoms in patients who have CRH overactivity, wherein the method may comprise the following steps:

(a) determining in a nucleic acid sample obtained from a patient the presence or absence of at least one single nucleotide polymorphism (SNP) associated with a value indicative for CRH overactivity;

(b) predicting the treatment response to CRHR1 antagonists by linking the algorithm provided by the method for providing an algorithm as described above with the presence or absence of the at least one SNP determined in step (b).

### BRIEF DESCRIPTION OF THE DRAWINGS

FIG. 1: Graph of the phenotypic distribution of ln(AAUC) at in-patient admission. The X-axis shows the ln of the AUC of the ACTH response and the Y-axis the frequency in total N/bin. The dashed vertical indicates the cut-off for being classified as a low vs. high responder. AAUC is the area under the curve of the ACTH response.

FIG. 2: Increased REMS activity in CRH-COE^{CNS} mice is suppressed by DMP696 (50 mg/kg/d) application via drinking water. Treatment day one, light grey; treatment day 2, dark grey; treatment day three, black. Symbols indicate significant differences between baseline and treatment day one (+), two (#) or three (*). Light and dark bar on the x-axis indicate light and dark period, respectively.

FIG. 3: Increased activity of REMS in CRH-COE^{CNS} is suppressed by application of the CRH-R1 antagonist SSR125543 (50 mg/kg/d) via drinking water. Baseline day, white; treatment day two, dark grey; treatment day three, black. Symbols indicate significant differences between baseline and treatment day two (#) or three (*). Light and dark bar on the x-axis indicate light and dark period, respectively.

FIG. 4: REMS activity in Cor26 CRH mice is suppressed by application of the CRH-R1 antagonist CP-316311 (50 mg/kg/d) via drinking water. Baseline day, white; treatment day two, dark grey; treatment day three, black.

### DETAILED DESCRIPTION OF THE INVENTION

Where the term “comprise” or “comprising” is used in the present description and claims, it does not exclude other elements or steps. For the purposes of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

Where an indefinite or a definite article is used when referring to a singular noun such as “a” or “an”, or “the”, this includes a plural form of that noun unless specifically stated.

Vice versa, when the plural form of a noun is used it refers also the singular form. For example, when SNPs are mentioned, this is also to be understood as a single SNP.

Furthermore, the terms first, second, third or (a), (b), (c) and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Further definitions of the terms will be given in the following in the context of which the terms are used.

It has been found that a treatment response to CRH antagonists in patients with depressive symptoms and/or anxiety symptoms can be reliably predicted by using a machine-learning based prediction algorithm derived from the association of SNPs with values indicative for CRH overactivity.

Further, a patient group responsive to the treatment with CRHR1 antagonists has been identified. In particular, patients with depressive and/or anxiety symptoms having CRH overactivity can be successfully treated with CRHR1 antagonists.

The term “treatment response to CRHR1 antagonists in patients with depressive symptoms and/or anxiety symptoms” in the sense of the invention refers to a response in a patient with depressive symptoms and/or anxiety symptoms during and/or after the treatment with one or more CRHR1 antagonists compared to before the treatment. The response may range from a partial alleviation of the symptoms to a complete remission of the symptoms, indicated by the change of symptoms strength and/or frequency of relapse of individual symptoms and/or the mean change on a depression scale, e.g. as described herein. The response can occur shortly after treatment or after a certain time period. A decrease in symptom severity from pretreatment of 25% or more is usually considered a partial alleviation of symptoms. Remission may be defined as achieving a value of 8 or less on the Hamilton Depression Rating Scale (HAM-D) or equivalent values on other rating scales named below.

The term “CRHR1 antagonist” refers to a compound capable of binding directly or indirectly to a CRH receptor 1 so as to modulate the receptor mediated activity. CRHR1 antagonists are well known in the literature and are e.g. described in WO 94/13676, EP 0 773 023, WO 2004/047866, WO 2004/094420, WO 98/03510, WO 97/029109, WO 2006/044958, WO 2001/005776 and WO 95/033750. Exemplary CRHR1 antagonists comprise NBI30775/R121919 (Neurocrine), CP316.311 (Pfizer), CP154,526 (Pfizer), Emicerfont (Glaxo), ONO-2333Ms (Ono Pharmaceutical), Pexacerfont (Bristol-Myers-Squibb), SSR125543 (Sanofi-Aventis), NBI-34101 (Neurocrine) and TAI041 (Taisho). Further exemplary CRHR1 antagonists comprise Antalarmin, CRA 5626, DMP-696, DMP-904, DMP-695, SC-241, BMS-561388, NBI30545, PD-171729, Verucerfont, NBI34041, NBI35965, SN003, CRA0450, CP-376,395, NBI-27914, PF-572778, GSK561579 and GSK586529.

One aspect concerns the provision of an algorithm for predicting a treatment response to CRHR1 antagonists in patients with depressive symptoms and/or anxiety symptoms. The method may comprise the following steps:

(a) performing a single nucleotide polymorphism (SNP) genotyping analysis in a group of patients with depressive symptoms and/or anxiety symptoms;

(b) determining a value indicative for CRH activity in each patient of the group, wherein a value indicative for CRH overactivity is indicative or predictive for a patient responding to a treatment with a CRH1 antagonist;

(c) identifying at least one SNP associated with a value indicative for CRH overactivity as determined in step (b);

(d) determining the algorithm by machine-learning from the association of the at least one SNP identified in step (c) with the value indicative for CRH overactivity.

In a step (a), a single nucleotide polymorphism (SNP) genotyping analysis in a group of patients with depressive symptoms and/or anxiety symptoms is performed.

Depressive symptoms comprise inter alia low mood, low self-esteem, loss of interest or pleasure, psychosis, poor concentration and memory, social isolation, psychomotor agitation/retardation, thoughts of death or suicide, significant weight change (loss/gain), fatigue, and feeling of worthless. The depressive disorders can last for weeks to lifelong disorder with periodic reoccurring depressive episodes. For the diagnosis of the depression mode (e.g. moderate or severe depression) the Hamilton Depression Rating Scale (HAM-D) (Hamilton, J Neurol Neurosurg Psychiatry, 1960) may be used. The depression mode may be also rated by alternative scales as the Beck Depression Inventory (BDI), the Montgomery-Åsberg Depression Scale (MADRS), the Geriatric Depression Scale (GDS), the Zung Self-Rating Depression Scale (ZSRDS).

Anxiety symptoms comprise inter alia panic disorders, generalized anxiety disorder, phobias and posttraumatic stress disorder. Typical symptoms of anxiety are avoidance behavior which may lead to social isolation, physical ailments like tachycardia, dizziness and sweating, mental apprehension, stress and tensions. The strength of these symptoms ranges from nervousness and discomfort to panic and terror in humans or animals. Most anxiety disorders may last for weeks or even months, some of them even for years and worsen if not suitably treated. For measuring the severity of anxiety symptoms, the Hamilton Anxiety Rating Scale (HAM-A) or the State-Trait Anxiety Rating Scale (STAI) can be used.

A “group of patients” as used herein comprises at least two patients, such as at least 10 patients, or at least 100 patients, or at least 150 patients. Patients included in the analysis of step (a) may exhibit at least a moderate to severe depressive mode. The group of patients may comprise patients with CRH overactivity and/or patients with normal CRH activity.

The term “polymorphism” as used herein refers to a variation in the genome of individuals, including, insertions, deletions, point mutations and translocations.

The term “single nucleotide polymorphism” is well understood by the skilled person and refers to a point mutation at a certain position in the nucleotide sequence. In other words, only one nucleotide differs in a certain region.

The nucleotide, that is present in the majority of the population, is also referred to as wild-type allele or major allele. As used herein, this state is defined as “absence of a SNP”.

The specific nucleotide that is present in the minority of the population is also referred as the point mutation, mutated nucleotide or minor allele. As used herein this state is defined as “presence of a SNP”.

The term “biomarker”, as used herein, relates to any nucleic acid sequence of any length, or a derivative thereof, which comprises a polymorphic variant as defined in:
SNP rs6437726,
SNP rs1986684,
SNP rs7380830,
SNP rs3903768,
SNP rs7325978,
SNP rs13585,
SNP rs9368373,
SNP rs10935354,
SNP rs8095703,
SNP rs10206851,
SNP rs9542977,
SNP rs4942879,
SNP rs9542954,
SNP rs1593478,
SNP rs9542951,
SNP rs2188534,
SNP rs12524124,
SNP rs4352629,
SNP rs7448716,
SNP rs11873533,
SNP rs10062658,
SNP rs12547917,
SNP rs1038268,
SNP rs2375811,
SNP rs1352671,
SNP rs364331,
SNP rs1924949,
SNP rs11025990,
SNP rs3758562, and/or
SNP rs10156056.

A biomarker may, for instance, be represented by a nucleic acid molecule of a length of e.g. 1 nt, 2 nt, 3 nt, 4 nt, 5 nt, 10 nt, 15 nt, 20 nt, 25 nt, 30 nt, 35 nt, 40 nt, 45 nt, 50 nt, 60 nt, 70 nt, 80 nt, 90 nt, 100 nt, 200 nt, 300 nt, 400 nt, 500 nt, 1000 nt, 2000 nt, or more or any length in between these lengths. The representing nucleic acid may be any suitable nucleic acid molecule, e.g. a DNA molecule, e.g. a genomic DNA molecule or a cDNA molecule, or a RNA molecule, or a derivative thereof. The biomarker may further be represented by translated forms of the nucleic acid, e.g. a peptide or protein as long as the polymorphic modification leads to a corresponding modification of the peptide or protein. Corresponding information may be readily available to the skilled person from databases such as the NCBI SNP repository and NCBI Genbank.

The SNPs as described herein may be present on the Watson or the Crick strand, with presence of the corresponding base. I.e. if, for example, a polymorphism is present on the Watson strand as A, it is present on the Crick strand as T, if the polymorphism is present on the Watson strand as T, it is present on the Crick strand as A, if the polymorphism is present on the Watson strand as G, it is present on the Crick strand as C, and if the polymorphism is present on the Watson strand as C, it is present on the Crick strand as G, and vice versa. Also the insertion or deletion of bases may be detected on the Watson and/or the Crick strand, with correspondence as defined above. For analytic purposes the strand identity may be defined, or fixed, or may be choose at will, e.g. in dependence on factors such the availability of binding elements, GC-content etc. Furthermore, for the sake of accuracy, the SNP may be defined on both strands (Crick and Watson) at the same time, and accordingly be analyzed.

A “polymorphic site” or “polymorphic variant” as used herein relates to the position of a polymorphism or SNP as described herein within the genome or portion of a genome of a subject, or within a genetic element derived from the genome or portion of a genome of a subject.

In theory, the wild-type allele could be mutated to three different nucleotides. However, the event of a mutation to a first nucleotide in the reproductive cells of an individual that gets established in a population occurs very rarely. The event that the same position is mutated to a second nucleotide and established in the population virtually never occurs and can be therefore neglected. Therefore, as used herein, a certain nucleotide position in the genome of an individual can have two states, the wild-type state (absence of a SNP) and the mutated state (presence of a SNP).

The term “wildtype sequence” as used herein refers to the sequence of an allele, which does not show the CRH overactivity phenotype according to the present invention. The term may further refer to the sequence of the non phenotype-associated allele with the highest prevalence within a population, e.g. within a Caucasian population.

The term “indicator sequence” as used herein refers to the sequence of an allele, which shows an association with a CRH overactivity phenotype according to the present invention. The term “indicator nucleotide” as used herein refers to the identity of the nucleotide at the position of a SNP or polymorphic site as defined herein, associated with a CRH overactivity phenotype according to the present invention. The term may refer to a non-wildtype nucleotide at positions of SEQ ID NO: 1 to 30 as described below:
SNP rs6437726 which is represented by a single polymorphic change at position 201 of SEQ ID NO: 1, wherein in one or two alleles the wild-type nucleotide A is replaced by indicator nucleotide G,
SNP rs1986684 which is represented by a single polymorphic change at position 201 of SEQ ID NO: 2, wherein in one or two alleles the wild-type nucleotide A is replaced by indicator nucleotide G,
SNP rs7380830 which is represented by a single polymorphic change at position 201 of SEQ ID NO: 3, wherein in one or two alleles the wild-type nucleotide C is replaced by indicator nucleotide T,
SNP rs3903768 which is represented by a single polymorphic change at position 201 of SEQ ID NO: 4, wherein in one or two alleles the wild-type nucleotide A is replaced by indicator nucleotide G,
SNP rs7325978 which is represented by a single polymorphic change at position 201 of SEQ ID NO: 5, wherein in one or two alleles the wild-type nucleotide C is replaced by indicator nucleotide T,
SNP rs13585 which is represented by a single polymorphic change at position 185 of SEQ ID NO: 6, wherein in one or two alleles the wild-type nucleotide C is replaced by indicator nucleotide T,
SNP rs9368373 which is represented by a single polymorphic change at position 201 of SEQ ID NO: 7, wherein in one or two alleles the wild-type nucleotide C is replaced by indicator nucleotide T,
SNP rs10935354 which is represented by a single polymorphic change at position 201 of SEQ ID NO: 8, wherein in one or two alleles the wild-type nucleotide A is replaced by indicator nucleotide G,
SNP rs8095703 which is represented by a single polymorphic change at position 201 of SEQ ID NO: 9, wherein in one or two alleles the wild-type nucleotide A is replaced by indicator nucleotide G,
SNP rs10206851 which is represented by a single polymorphic change at position 201 of SEQ ID NO: 10, wherein in one or two alleles the wild-type nucleotide C is replaced by indicator nucleotide T,
SNP rs9542977 which is represented by a single polymorphic change at position 201 of SEQ ID NO: 11, wherein in one or two alleles the wild-type nucleotide C is replaced by indicator nucleotide T,
SNP rs4942879 which is represented by a single polymorphic change at position 201 of SEQ ID NO: 12, wherein in one or two alleles the wild-type nucleotide A is replaced by indicator nucleotide G,
SNP rs9542954 which is represented by a single polymorphic change at position 201 of SEQ ID NO: 13, wherein in one or two alleles the wild-type nucleotide A is replaced by indicator nucleotide C,
SNP rs1593478 which is represented by a single polymorphic change at position 201 of SEQ ID NO: 14, wherein in one or two alleles the wild-type nucleotide C is replaced by indicator nucleotide T,
SNP rs9542951 which is represented by a single polymorphic change at position 201 of SEQ ID NO: 15, wherein in one or two alleles the wild-type nucleotide A is replaced by indicator nucleotide G,
SNP rs2188534 which is represented by a single polymorphic change at position 200 of SEQ ID NO: 16, wherein in one or two alleles the wild-type nucleotide G is replaced by indicator nucleotide T,
SNP rs12524124 which is represented by a single polymorphic change at position 201 of SEQ ID NO: 17, wherein in one or two alleles the wild-type nucleotide A is replaced by indicator nucleotide G,
SNP rs4352629 which is represented by a single polymorphic change at position 201 of SEQ ID NO: 18, wherein in one or two alleles the wild-type nucleotide C is replaced by indicator nucleotide T,
SNP rs7448716 which is represented by a single polymorphic change at position 201 of SEQ ID NO: 19, wherein in one or two alleles the wild-type nucleotide A is replaced by indicator nucleotide G,
SNP rs11873533 which is represented by a single polymorphic change at position 201 of SEQ ID NO: 20, wherein in one or two alleles the wild-type nucleotide A is replaced by indicator nucleotide C,
SNP rs10062658 which is represented by a single polymorphic change at position 201 of SEQ ID NO: 21, wherein in one or two alleles the wild-type nucleotide A is replaced by indicator nucleotide G,
SNP rs12547917 which is represented by a single polymorphic change at position 201 of SEQ ID NO: 22, wherein in one or two alleles the wild-type nucleotide C is replaced by indicator nucleotide T,
SNP rs1038268 which is represented by a single polymorphic change at position 201 of SEQ ID NO: 23, wherein in one or two alleles the wild-type nucleotide C is replaced by indicator nucleotide T,
SNP rs2375811 which is represented by a single polymorphic change at position 201 of SEQ ID NO: 24, wherein in one or two alleles the wild-type nucleotide A is replaced by indicator nucleotide G,
SNP rs1352671 which is represented by a single polymorphic change at position 201 of SEQ ID NO: 25, wherein in one or two alleles the wild-type nucleotide A is replaced by indicator nucleotide C,
SNP rs364331 which is represented by a single polymorphic change at position 201 of SEQ ID NO: 26, wherein in one or two alleles the wild-type nucleotide A is replaced by indicator nucleotide C,
SNP rs1924949 which is represented by a single polymorphic change at position 201 of SEQ ID NO: 27, wherein in one or two alleles the wild-type nucleotide A is replaced by indicator nucleotide G,
SNP rs11025990 which is represented by a single polymorphic change at position 201 of SEQ ID NO: 28, wherein in one or two alleles the wild-type nucleotide A is replaced by indicator nucleotide G,
SNP rs3758562 which is represented by a single polymorphic change at position 201 of SEQ ID NO: 29, wherein in one or two alleles the wild-type nucleotide A is replaced by indicator nucleotide G,
SNP rs10156056 which is represented by a single polymorphic change at position 201 of SEQ ID NO: 30, wherein in one or two alleles the wild-type nucleotide C is replaced by indicator nucleotide G.

The term “allele” or “allelic sequence” as used herein refers to a particular form of a gene or a particular nucleotide, e.g. a DNA sequence at a specific chromosomal location or locus. In certain embodiments of the present invention a SNP as defined herein may be found at or on one of two alleles in the human genome of a single subject. In further, specific embodiments, a SNP as defined herein may also be found at or on both alleles in the human genome of a single subject. The presence of an indicator nucleotide or an indicator triplet as defined herein on both alleles may have a higher predictive value than the presence of an indicator nucleotide or an indicator triplet on one allele only, the other allele comprising a wildtype genotype. The presence or absence of indicator nucleotides or indicator triplets on one or two alleles may be connected or linked with an algorithm for predicting the a treatment response to CRHR1 antagonists in patients with depressive symptoms and/or anxiety symptoms as described herein.

A person skilled in the art would be able to derive the exact position, nucleotide sequence, and indicator sequence from the above identified rs-nomenclature, e.g. from suitable database entries and associated information systems, e.g. the Single Nucleotide Polymorphism database (dbSNP) which is incorporated herein by reference. The information may also be retrievable in case of changes to the nomenclature, or to the surrounding sequence elements, e.g. based on history functions of a suitable database.

The term “isolated nucleic acid molecule” a used herein refers to a nucleic acid entity, e.g. DNA, RNA etc, wherein the entity is substantially free of other biological molecules, such as, proteins, lipids, carbohydrates, other nucleic acids or other material, such as cellular debris and growth media. Generally, the term “isolated” is not intended to refer to the complete absence of such material, or to the absence of water, buffers, or salts, unless they are present in amounts which substantially interfere with the methods of the present invention.

The term “single nucleotide polymorphism (SNP) genotyping analysis” as used herein refers to a test of determining in one or several patients the presence or absence of at least one SNP, typically several SNPs, and in some embodiments all (known) SNPs the human genome, including endogenous and exogenous regions. In particular, a SNP genotyping analysis as used herein may not be limited to the CRHR1 gene or to genes of the CRH pathway. In other words, an SNP genotyping analysis as used herein can be a genome-wide screening for SNPs.

SNP genotyping analysis can be performed by methods known in the art such as microarray analysis or sequencing analysis or PCR related methods or mass spectrometry or 5′-nuclease assays or allele specific hybridization or high-throughput variants of these techniques or combinations thereof. These and other methods are known in the art. See for example Rampal, DNA Arrays: Methods and Protocols (Methods in Molecular Biology) 2010; Graham & Hill, DNA Sequencing Protocols (Methods in Molecular Biology) 2001; Schuster, Nat. Methods, 2008 and Brenner, Nat. Biotech., 2000; Mardis, Annu Rev Genomics Hum Genet., 2008. Genomewide arrays can be purchased from different suppliers such as Illumia and Affymetix.

For example, the determination of the nucleotide sequence and/or molecular structure may be carried out through allele-specific oligonucleotide (ASO)-dot blot analysis, primer extension assays, iPLEX SNP genotyping, Dynamic allele-specific hybridization (DASH) genotyping, the use of molecular beacons, tetra primer ARMS PCR, a flap endonuclease invader assay, an oligonucleotide ligase assay, PCR-single strand conformation polymorphism (SSCP) analysis, quantitative real-time PCR assay, SNP microarray based analysis, restriction enzyme fragment length polymorphism (RFLP) analysis, targeted resequencing analysis and/or whole genome sequencing analysis.

In some embodiments, any of the methods described herein comprises the determination of the haplotype for two copies of the chromosome comprising the SNPs identified herein.

The term “determining the status of a biomarker” as used herein refers to any suitable method or technique of detecting the identity of an SNP, e.g. at the positions of the biomarkers described herein. The determination method may be a sequencing technique or a technique based on complementary nucleic acid binding. The context of the indicated positions, as well as the strand may differ, e.g. from patient to patient, or from sample to sample etc.

A “subject's sample” as used herein may be any sample derived from any suitable part or portion of a subject's body. In some embodiments, blood or saliva samples are used. The sample used in the context of the present invention should be collected in a clinically acceptable manner, in particular in a way that nucleic acids and/or proteins are preserved.

The term “primer” may denote an oligonucleotide that acts as an initiation point of nucleotide synthesis under conditions in which synthesis of a primer extension product complementary to a nucleic acid strand is induced.

The term “probe” may denote an oligonucleotide that selectively hybridizes to a target nucleic acid under suitable conditions.

The primers and probes may be generated such that they are able to discriminate between wild-type allele or mutated allele of the position of a SNP to be analyzed. Methods for the design of sequence specific primers and probes are known in the art (see e.g. William B. Coleman, Gregory J. Tsongalis, Molecular Diagnostics: For the Clinical Laboratorian, 2007; Weiner et al. Genetic Variation: A Laboratory Manual, 2010).

Typically, a SNP is considered in the genotyping analysis if it occurs in a certain percentage in the population, for example in at least 5% or at least 10% of the population. In other words, the minor allele frequency (MAF) is larger than 0.05 or 0.10 (MAF>0.05 or MAF>0.10).

For the SNP genotyping analysis a nucleic acid or DNA sample from a patient may be used. The nucleic acid or DNA sample can be a blood sample, a hair sample, a skin sample or a salvia sample of the patient. Any other sample obtainable from the patient and containing patient nucleic acid or DNA can also be used. The sample can be collected from the patient by any method known in the art. For example, a blood sample can be taken from a patient by use of a sterile needle. The collection of salvia out of the mouth and throat of the patient can be performed by use of a sterile cotton bud or by flushing said area and collecting the flushing solution.

Usually, the nucleic acid or DNA is extracted or isolated or purified from the sample prior to SNP genotyping analysis. Any method known in the art may be used for nucleic acid or DNA extraction or isolation or purification. Suitable methods comprise inter alia steps such as centrifugation steps, precipitation steps, chromatography steps, dialyzing steps, heating steps, cooling steps and/or denaturation steps. For some embodiments, a certain nucleic acid or DNA content in the sample may be reached. Nucleic acid or DNA content can be measured for example via UV spectrometry as described in the literature. However, in alternative embodiments SNP genotyping analysis may also be performed by using a non-extracted or non-purified sample.

Nucleic acid or DNA amplification may also be useful prior to the SNP analysis step. Any method known in the art can be used for nucleic acid or DNA amplification.

The sample can thus be provided in a concentration and solution appropriate for the SNP analysis.

The analyzed SNPs may be represented by values 0, 1 or 2. The value “0” may indicate that the SNP is present on none of the two homologous chromosomes. The value “1” may indicate that the SNP is present on one of the two homologous chromosomes. The value “2” may indicate that the SNP is present on both homologous chromosomes. Homologous chromosomes correspond to each other in terms of chromosome length, gene loci and staining pattern. One is inherited from the mother, the other is inherited from the father.

In a step (b) of the method for providing a prediction algorithm, a value indicative for CRH activity in each patient is determined.

The term “normal CRH activity” refers to a range of CRH activity which can be found in human beings who are not affected by a condition which is associated with an increase of CRH activity (such as depression). A value indicative for normal CRH activity is usually considered to be indicative or predictive for a patient not responding to a treatment with a CRH1 antagonist.

The terms “CRH overactivity”, “CRH system overactivity”, “CRH hyperactivity”, “CRH hyperdrive” or “central CRH hyperdrive” are used herein interchangeable. An indication for CRH overactivity may be an increase in activity or concentration of CRH or of one or several molecules downstream of the CRHR1 receptor, that are activated or whose concentration is increased based on the activation of CRHR1 receptor upon CRH binding. A further indication for CRH overactivity may be a decrease in activity or concentration of one or several molecules downstream of the CRHR1 receptor, that are inactivated or whose concentration is decreased based on the activation of CRHR1 receptor upon CRH binding. A value indicative for CRH overactivity is usually considered to be indicative or predictive for a patient responding to a treatment with a CRHR1 antagonist. CRH activity vs. CRH overactivity may be defined relatively to the whole group, e.g. by using a median split of the area under the curve of the ACTH response in the dex/CRH test. Responses in the upper median may be categorized as being predictive of CRH overactivity, while responses in the lower median are indicative of normal CRH activity.

A “value indicative for CRH activity”, a “value indicative for CRH overactivity” and/or a “value indicative for normal CRH activity” can be obtained by determining the concentration or activity of CRH and/or of a downstream target of the CRHR1 receptor. The analysis is usually set up in a way that it can be excluded that the modulation of activity or concentration of a downstream target of the CRHR1 receptor is due to another disturbance than CRH activity. For instance, the concentrations or activities of adrenocorticotrophin (ACTH) and/or cortisol are useful biomarkers for determining a value indicative for CRH overactivity.

Typically, the CRH overactivity in each patient may be determined by measuring the ACTH and/or cortisol level response to a combined dexamethasone suppression/CRH stimulation test as described in Heuser et al. (J Psychiatr Res., 1994). In one embodiment of a combined dexamethasone suppression CRH stimulation test, subjects are pre-treated with dexamethasone and blood is drawn in certain intervals. Further, human CRH is administered after the first pretreatment with dexamethasone. Plasma ACTH and/or cortisol concentrations are then determined. The neuroendocrine response to the dex/CRH test may be analyzed using the total area under the curve (AUC) of the ACTH response. Details of an exemplary dex/CRH test are also described in Example 1 below.

The neuroendocrine response to the dex/CRH test may be analyzed using the total area under the curve (AUC) of the ACTH response. Patients suffering from depression normally show an increased release of cortisol and of adrenocorticotropic hormone (ACTH) in response to the combined treatment with dexamethasone and CRH as performed during the test, thus indicating a dysregulation of the hypothalamic-pituitary-adrenal (HPA) axis. Patients with a high HPA axis dysregulation show AUC values of cortisol of between 3000 and 18000 AUC units (ng/ml×75 min) and/or AUC values of ACTH of between 1000 and 6500 AUC units (pg/ml×75 min). Patients having a low HPA axis dysregulation show AUC values of cortisol of between 300 and 2500 AUC units (ng/ml×75 min) and/or AUC values of ACTH of between 250 and 1000 AUC units (pg/ml×75 min) Various antidepressants lead to a reduction of these increased cortisol and ACTH levels in a combined dex/CRH test performed after the treatment with the antidepressants. Treatment response to antidepressants can thus be determined by performing a second dex/CRH test after treatment with the antidepressant and comparing the neuroendocrine response to the one shown in a combined dex/CRH test performed prior to treatment with the antidepressant.

The term “normal CRH activity” refers to a range of CRH activity which can be found in human beings who are not affected by a condition which is associated with an increase of CRH activity (such as depression). A value indicative for normal CRH activity is usually considered to be indicative or predictive for a patient not responding to a treatment with a CRHR1 antagonist.

“Downstream target” or “molecule which is downstream of the CRHR1 receptor” as used herein may denote a molecule such as an endogenous molecule (e.g. peptide, protein, lipid, nucleic acid or oligonucleotide) that is regulated by CRHR1 directly or indirectly. The direct or indirect regulation may comprise direct or indirect modulation of the activity and/or expression level and/or localization, degradation, stability of the downstream target.

Steps (c) and (d) of the method for providing a prediction algorithm may analyze the association of the analyzed SNPs with the value indicative for CRH overactivity and/or normal CRH activity and generate an algorithm for predicting the treatment response to CRHR1 antagonists.

In an exemplary embodiment, the group of patients may be split into two sets of similar size and similar values for descriptors such as demographic descriptors or clinical descriptors. These two sets are hereinafter also referred to as “training set” and “test set”.

In step (c) of the method of this exemplary embodiment, at least one SNP associated with the value indicative for CRH overactivity as determined in step (b) is identified in the training set.

Further, there can be at least two alternatives for the result provided by the prediction algorithm. First, the result may be a categorical answer whether the patient responds to CRHR1 antagonist treatment or not. Second, the prediction algorithm may provide the answer to which degree the patient responds or does not respond to the treatment. Depending on the desired result provided by the prediction algorithm the way of determining the algorithm may differ.

In the alternative that the prediction algorithm will analyze whether a patient responds or does not respond to CRHR1 antagonist treatment, the values indicative for CRH activity may be provided as logic data variable (Boolean type; 0 vs. 1; true vs. false, high vs. low responder). Therefore, if the test performed to determine values indicative for CRH overactivity provides a data range, the patients may be dichotomized by a threshold into high vs. low responders.

In the alternative that the test will analyze to which degree the patient responds or does not respond to the CRHR1 antagonist treatment, the values indicative for CRH activity may be provided as numerical values.

Typically, SNPs that are modified in a significant percentage of the population are used in the method for providing a prediction algorithm. For example, only SNPs with a minor allele frequency (MAF) greater than 0.05 or 0.10 may be selected for further analysis. This means that only SNPs that are modified in at least 5% or 10% of the population are selected for further analysis.

Association for all SNPs with the value indicative for CRH activity is tested by an association analysis testing the likelihood for a patient to be CRH overactive vs. CRH non-overactive in dependence of the genotype of said patient. Said association analysis may be conducted for example by an additive genetic model and/or by a logistic regression. A SNP is e.g. identified to be associated with a value indicative for CRH overactivity if the corresponding p-value is at least 1×10⁻³ or at least 1×10⁻⁴ or at least 1×10⁻⁵. The lower the p-value the more the SNP is associated with a value indicative for CRH overactivity. Accordingly, a SNP is e.g. identified to be associated with a value indicative for normal CRH activity if the corresponding p-value is at least 1×10⁻³ or at least 1×10⁻⁴ or at least 1×10⁻⁵.

In the step (d) of this exemplary embodiment, the algorithm for predicting a treatment response to CRHR1 antagonists may be determined by the use of SNPs in the test set by a machine learning method.

The term “algorithm for predicting” as used herein may refer to a classification function (also known as binary classification test).

The term “machine-learning” as used herein may refer to a method known to the person skilled in the art of machine learning. In particular, machine learning is concerned with the design and development of algorithms that allow computers to evolve behaviors based on empirical data, such as from sensor data or databases. It may be selected from the group consisting of artificial neural network learning, decision tree learning, support vector machine learning, Bayesian network learning, clustering, and regression analysis.

The term “reliable prediction of the treatment response to CRHR1 antagonists” as used herein may refer to a high performance of the prediction algorithm. The evaluation of the performance of the prediction algorithm may depend on the problem the algorithm is applied for. If the algorithm is used to identify patients that are likely to response to the treatment with CRHR1 antagonists the performance is usually expressed by a high accuracy and/or sensitivity and/or precision. If patients should be identified which are likely not to respond to the treatment with CRHR1 antagonists, specificity and/or negative predictive value are typical statistical measures to describe the performance of the prediction algorithm.

For optimizing the prediction performance of the algorithm, the step of determining the algorithm by a machine-learning method in a first subset of the test set and testing the prediction performance in an second independent subset of the test set may be repeated based on different numbers and groups of SNPs, until the desired prediction performance is reached.

Accuracy, sensitivity, precision, specificity and negative predictive value are exemplary statistical measure of the performance of the prediction algorithm. In the following, examples are given for determining the performance of the prediction algorithm.

As used herein, accuracy may be calculated as (number of true positives+number of true negatives)/(number of true positives+number of false positives+number of true negatives+number of false negatives), e.g. (number of patients correctly diagnosed as responding to CRHR1 antagonist+number of patients correctly diagnosed as not responding to CRHR1 antagonist)/(number of patients correctly diagnosed as responding to CRHR1 antagonist+number of patients wrongly diagnosed as responding to CRHR1 antagonist+number of patients correctly diagnosed as not responding to CRHR1 antagonist+number of patients wrongly diagnosed as not responding to CRHR1 antagonist). The accuracy of prediction may e.g. be at least 60%, at least 70%, at least 80% or at least 90%.

A used herein, sensitivity may be calculated as (true positives)/(true positives+false negatives), e.g.: (number of patients correctly diagnosed as responding to CRHR1 antagonist)/(number of patients correctly diagnosed as responding to CRHR1 antagonist+number of patients wrongly diagnosed as not responding to CRHR1 antagonist). The sensitivity of prediction may be at least 60%, at least 70%, at least 80% or at least 90%.

As used herein, precision (also referred to as positive predictive value) may be calculated as (true positives)/(true positives+false positives), e.g.: (number of patients correctly diagnosed as responding to CRHR1 antagonist)/(number of patients correctly diagnosed as responding to CRHR1 antagonist+number of patients wrongly diagnosed as responding to CRHR1 antagonist). The precision of prediction may be at least 60%, at least 70%, at least 80% or at least 90%.

As used herein, specificity is calculated as (true negatives)/(true negatives+false positives), e.g.: (number of patients correctly diagnosed as not responding to CRHR1 antagonist)/(number of patients correctly diagnosed as not responding to CRHR1 antagonist+number of patients wrongly diagnosed as responding to CRHR1 antagonist). The specificity of prediction may be at least 60%, at least 70%, at least 80% or at least 90%.

As used herein, negative predictive value is calculated as (true negatives)/(true negatives+false negatives), e.g.: (number of patients correctly diagnosed as not responding to CRHR1 antagonist)/(number of patients correctly diagnosed as not responding to CRHR1 antagonist+number of patients wrongly diagnosed as not responding to CRHR1 antagonist). The negative predictive value may be at least 60%, at least 70%, at least 80% or at least 90%.

Other statistical measures useful for describing the performance of the prediction algorithm are geometric mean of sensitivity and specificity, geometric mean of positive predictive value and negative predictive value, F-measure and area under ROC curve, and the positive and negative likelihood ratios, the false discovery rate and Matthews correlation coefficient. These measures and method for their determination are well known in the art.

In general, a prediction algorithm with high sensitivity may have low specificity and vice versa. The decision to select an algorithm having certain statistical characteristics such as accuracy, sensitivity or specificity may also depend on the costs associated with a treatment with a CRHR1 antagonist should the prediction be positive and/or whether such a treatment is detrimental in cases where the result is a false positive.

For a prediction whether a patient likely responds to the treatment with CRHR1 antagonists the prediction algorithm may be based on a number of SNPs sufficient to achieve a prediction sensitivity and/or precision of at least 55%, optionally at least 80%.

For the prediction whether it is unlikely that a patient responds to the treatment with CRHR1 antagonists the prediction algorithm may be based on a number of SNPs sufficient to achieve a prediction specificity and/or negative predictive value of at least 55%, optionally at least 80%.

For a prediction whether a patient responds to a treatment with CRHR1 antagonists or not the prediction algorithm may be based on a number of SNPs sufficient to achieve sensitivity and/or precision and/or specificity and/or negative predictive value of at least 55%, optionally at least 80%.

In one embodiment, a number N of SNPs is associated with a value indicative for CRH overactivity or normal CRH activity in step (d) of the method for providing an algorithm and/or the presence or absence of a number N of SNPs is determined in step (a) of the method for predicting a treatment response, wherein N is sufficient to provide an accuracy of at least 80% and a sensitivity of at least 70% and a specificity of at least 70%. In another embodiment, a number N of SNPs is associated with a value indicative for CRH overactivity in step (d) of the method for providing an algorithm and/or the presence or absence of a number N of SNPs is determined in step (a) of the method for predicting a treatment response, wherein N is sufficient to provide an accuracy of at least 85% and a sensitivity of at least 80% and a specificity of at least 80%.

Typically, at least 10, at least 20, at least 25 or at least 30 SNPs are used for determination of the algorithm in step (d) of the method for providing a prediction algorithm.

The skilled person in the art knows that the use of different machine-learning methods and adapting parameters used therein can be also used for improvement of the prediction reliability. The whole statistical work-flow can be automated by a computer.

Another aspect of the invention is a method for predicting a treatment response to CRHR1 antagonists in patients with depressive symptoms and/or anxiety symptoms, wherein the method may comprise the following steps:

(a) determining in a nucleic acid sample obtained from a patient the presence or absence of at least one single nucleotide polymorphism (SNP) associated with a value indicative for CRH overactivity;

(b) predicting the treatment response to CRHR1 antagonists by linking the algorithm provided by the method of claim 1 with the presence or absence of the at least one SNP determined in step (a).

“Linking an algorithm for predicting a treatment response to CRHR1 antagonists in patients having depressive symptoms and/or anxiety symptoms with the presence or absence of the at least one SNP” as used herein may refer to using such an algorithm to predict the treatment response based on the determined presence or absence of the at least one SNP, e.g. by integrating the at least one SNP determined in step (a) of the above method by the algorithm.

In one embodiment of the method for predicting a treatment response to CRHR1 antagonists in patients with depressive symptoms and/or anxiety symptoms, step (a) comprises determining at least one of the SNPs, optionally all of the SNPs which were associated with a value indicative for CRH overactivity when determining the algorithm by machine-learning from this association.

In another embodiment of the method for predicting a treatment response to CRHR1 antagonists in patients with depressive symptoms and/or anxiety symptoms, the method may be accompanied by analyzing the rapid-eye-movement (REM) during night sleep of a patient in a sleep EEG.

REM sleep typically comprises a characteristic coincidence of nearly complete muscle atonia, a waking-like pattern of brain oscillations and rapid eye movements (REMs). The amount of REMs during consecutive REM sleep episodes is usually increasing throughout the night. Single and short REMs with low amplitude can be characteristic for initial parts of REM sleep. The amount of REMs in particular within the first REM sleep episode can be of clinical relevance. Recent clinical and animal data supports the correlation of REM density with an increased CRH activity. For example, Kimura et al. (Mol. Psychiatry, 2010) showed that mice overexpressing CRH in the forebrain exhibit constantly increased rapid eye movement (REM) sleep compared to wildtype mice. In addition, it could be shown that treatment with the CRHR1 antagonist DMP696 could reverse the REM enhancement. Thus, the SNP analysis and REM density analysis as described herein may be combined for predicting the response of patients with depressive symptoms and/or anxiety symptoms to treatment with a CRHR1 antagonist. The REM analysis may be carried out before, concomitant or after the SNP analysis. For example, the REM density analysis may be carried out on persons that where identified by the SNP analysis as CRH hyperdrive patients.

The recording of a “sleep-EEG” (also referred to “polysomatic recordings”) may comprise electroencephalography (EEG), vertical and horizontal electrooculography (EOG), electromyography (EMG) and/or electrocardiography (ECG). In EOG, muscle activities of right and left eye may be recorded by electrooculograms (one or typically two channels) in order to visualize the phasic components of REM sleep.

“REM analysis” or “analyzing the rapid-eye-movement (REM)” may refer to a method comprising recoding of muscle activities of right and left eye by EOG and then analyzing the electrooculogram. The recognition of REM in the electrooculogram may be done manually (for example by standard guidelines Rechtschaffen and Kales, 1968, Bethesda, Md.: National Institute of Neurological Diseases and Blindness).

In one embodiment the method comprises a further step of obtaining a nucleic acid sample from a patient preceding the step of SNP determination.

Typically, a SNP is considered in the genotyping analysis of step (a) of the method of prediction if it occurs in a certain percentage in the population, for example in at least 5% or at least 10% of the population. In other words, the minor allele frequency (MAF) is larger than 0.05 (MAF>0.05) or 0.10 (MAF>0.10).

For the SNP genotyping analysis of step (a) of the method of prediction a nucleic acid or DNA sample from a patient may be used. The nucleic acid or DNA sample can be a blood sample, a hair sample, a skin sample or a salvia sample of the patient. Any other sample of the patient containing nucleic acid or DNA can also be used. The sample can be collected from the patient by any method known in the art. For example, a blood sample can be taken from a patient by use of a sterile needle. The collection of salvia out of the mouth and throat of the patient can be performed by use of a sterile cotton bud or by flushing said area and collecting the flushing solution.

Usually, the nucleic acid or DNA is extracted or isolated or purified from the sample prior to SNP genotyping analysis. Any method known in the art may be used for nucleic acid or DNA extraction or isolation or purification. Suitable methods comprise inter alia steps such as centrifugation steps, precipitation steps, chromatography steps, dialyzing steps, heating steps, cooling steps and/or denaturation steps. For some embodiments, a certain nucleic acid or DNA content in the sample may be reached. Nucleic acid or DNA content can be measured for example via UV spectrometry as known in art. However, the SNP genotyping analysis may also be performed by using a non-extracted or non-purified sample.

Nucleic acid or DNA amplification may also be useful prior to the SNP analysis step. Any method known in the art can be used for nucleic acid or DNA amplification. The sampled can thus be provided in a concentration and solution appropriate for the SNP analysis.

The analyzed SNPs may be represented by values 0, 1 or 2. The value “0” may indicate that the SNP is present on none of the two homologous chromosomes. The value “1” may indicate that the SNP is present on one of the two homologous chromosomes. The value “2” may indicate that the SNP is present on both homologous chromosomes. Homologous chromosomes correspond to each other in terms of chromosome length, gene loci and staining pattern. One is inherited from the mother, the other is inherited from the father.

In order to determine SNPs, SNP-specific primers and/or probes, a primer extension reaction, SNP microarrays, DNA-sequencing may be used. These reagents and methods are routinely used in the art (see for example Chen, PCR Cloning Protocols (Methods in Molecular Biology) 2002; Schuster, Nat. Methods, 2008, Rampal, DNA Arrays: Methods and Protocols (Methods in Molecular Biology) 2010; Brenner, Nat. Biotech., 2000; Mardis, Annu Rev Genomics Hum Genet., 2008). For example, MALDI-TOF (matrix-assisted laser desorption ionization time of flight) mass spectrometry on the Sequenom platform (San Diego, USA) may be used to genotype the selected variants. For primer selection, multiplexing and assay design, and the mass-extension for producing primer extension products the MassARRAY Assay Designer software may be used using the sequences presented in table 1 as input. The MassARRAY Typer 3.4 software may be used for genotype calling. Any other reagent or method known to the person skilled in the art may be used in order to detect the SNPs in an individual.

In one embodiment, at least one SNP determined in step (a) and used for the prediction algorithm of step (b) is a SNP selected from the group consisting of SNPs as shown in table 1 and an SNP in strong linkage disequilibrium with any of the SNPs shown in table 1.

TABLE 1
SNPs (together with flanking sequences) which may be used
to predict the response to CRHR1 antagonists in patients
with depressive symptoms and/or anxiety symptoms. The
position of the SNP is indicated in bold as [wild-type
allele/mutated allele].

| SNP_ID | SEQUENCE |
|---|---|
| RS6437726 | CAAGAAAGAGAGTAATAAAAATAACCACAATGAGGGCTCTCATTAATACT |
| SEQ ID | GGATCTTATGGAAACCAATTGTTCAGTCCCTCAACAAAAGACCAGATGG |
| NO: 1 | GCAGGAAGCTAAATATACACCATGCACTAAACATTATGAGTATCATAGTT |
| | TACAAGTCAAAGGGGGCTCTATTGAAGATAGTTCTATTTTCCCTCTATAT |
| | T[A/G]TCTGCTAGACAATACCTGATAACATTATCCAAGTAAATGACAACTT |
| | GATAAATAGTAATTTCCAATGGTGAACAGAGGTGACATTTCCTCATTACA |
| | AAAATATTTTCTTTGGCAGATGAGATTAACTGAATAAGAAATCCACTGAC |
| | ACTGAAATCACAGAGCCAAATTCCCTATCACAGCACTTATCACATTGCGT |
| | TAGG |
| RS1986684 | TTCCTTGTAGCGGGGAGAGAGACTCAGGGAAGGCAGGGTGATACCTGA |
| SEQ ID | GTTGGGGCTTAAAGCAAGGTAGGGTGTGTGTGGTGATGGCAAAATAGG |
| NO: 2 | TAGGAAGACAGCACGGGCAAAGTCCTGGAGGCAAGGACAGAAAGAGGA |
| | AGTGGCAGGAAGTGAGGCTGGGGAAATGAGTAGGGGTCAATCATGATG |
| | TTTCTGGT[A/G]TAGGGAAGAGTTTGGAATGCATCCTCTAGGCCATACGC |
| | CATTGGGGGCTTTTAAGAAAGACAGTGATGTTGGTTTGATTTGCATTTTA |
| | TATAGACTTTTCTGGCAGCTGAGAGGAAGGTGGTTTTGAGAATCACAAA |
| | GCTGCGGGAAGATCAGTCAGGAGGGTTCTAGAATAATCCAGGCAAGAG |
| | CTGATGGGGACTGAG |
| RS7380830 | ACAGGGGTGGCTACTCTTTCTCCAGAAATAGGTGTCCTGTGGGGCATTT |
| SEQ ID | TGAAGTAGAATGTTGATAGTTGCTTTCAATTTTAGACTGGTAAATAAGAAT |
| NO: 3 | TGGGCATTTGAATTTCAATATACTCACTGTGTAACTGTTATTGAGTATGCT |
| | TTAAGTGACCTATAATACTGCTTCATTTAACTTTATTGTCCTAATAACT[C/ |
| | T]TCTTAGAGTGACAATAACTTAGGTTAGCCACTTGCCTAGGGTTCTGAA |
| | ACCAAGTAAATGGTGGAGCTGGAATTGCTGTTCTTGTCAGTCATTAGACT |
| | AGATCGGTTTTCTTCTTCCTACAAATTTTATATACTAAAAAATTTTGAAAAA |
| | AGACATTTTTCTTTGGGAAAAATAGGGAATGTCAGATCCCTTTGGAGATG |
| RS3903768 | CTCGCAGCAACCAAGCCTGCCCAAGCCGGGGAAACCTGGGGAGCAAAC |
| SEQ ID | CTTCACCTGCACTGTACATCAGAGACCAGTTGGCCCTATTTTGGCTCCT |
| NO: 4 | GTGGACAGGTAAGTATCCCTTTTGACTCATCCCCCAAATATCAGGTGAG |
| | CCAGGAAAATAAGGCCTTTGGCTTAGACAGTCAATTCAAAGTCTGCCAT |
| | AGCAT[A/C]CCTAATTACATCCCTATTGCCCCTTTTCTAGGTCGTTTCTCC |
| | TCTAACACGATTTTATTTTTCTGTCAGCCATTTTATTTTATTTCTCACCTTG |
| | AAATATATGTTTTCTTTGCAGTTTTTGCTTTGGCTTCCTGCTAACTCTATT |
| | TGGGCAATTGTTTAAGGCTGAACACTTGGTTATGAGAGGTACCCTGTTG |
| | TGTTGA |
| RS7325978 | TCATCAAGTCTCCTTTTTCTCTAGGAAAAATAACATTGTCAAGGTTATTAA |
| SEQ ID | CAGTCAATAAGCTGTCATAGGCTCAGCATGGATGGGGATATTGGGTTTC |
| NO: 5 | CTTGTGCTTATATGAAAGATGGGAAAATCCGAAGTTCTTTTCACCCTGAT |
| | ATGGAAAATACCCAACATGAGGAGAAGCAGCAGCTATATGATTCTGAGC |
| | A[C/T]AGAATGGGAGTAAGAATAGGGTCATGCTGTACTGATTATCTGCTA |
| | ATAAAATGCAAAAGTGTTAGGTAATTTCATCAATATCCAGTTAATACTAAT |
| | ATAGTTAATATTTCATGACTGGGTAATATTTTATAATGATAAATATTTTTAT |
| | AGATCTTAGCTCTTTTTATTCTCATATCAACTGTATGAAATCAGTGATTGG |
| | T |
| RS13585 | CTGGGGACCTCAGGGAGAGGTACGCAGGTTGCCATGGCTGCGTCTGCA |
| SEQ ID | GTCCACCTGCCTTTCCACGCCAGGGAGTCAGTGATGTGGAGCCCCCTG |
| NO: 6 | GGCCCCAGTGGAAGCAGCGATCAGACTATGTGTCCTTGAAATAATGTTT |
| | ATTCCACGCTGTCCCGACAGCCCCCTCTGCAGGTCCCCT[C/T]GGTGTA |
| | CTCTGAGGTGGGAAACCCTCCCTGGGGGCGGTGAAGGGGAACTCGGG |
| | CCACCCCACCAGCCAGCAGATGCTCCAGCAGCCAGAGCCCCAGCCTGG |
| | AGCTGAGGCTCTTCCTGGGGCTCGCCGGGCCCCTGCAGGCTTTTCGGA |
| | CCCTCAGCCAGCCCGGCTTCCTCTGCTTTGGGCAGCAGCAAGCTGGCC |
| | CTT |
| RS9368373 | TTCCTGTGCCTCAGCTCCTCTGTAGAATGGTGCTGGCAATACAGTTTGC |
| SEQ ID | CTCATTGGGCTCTTGTAAGCTTTAAATAGGTTATTATACATAAAGAGCTA |
| NO: 7 | ATAGTGATGCCTGTAGCCGTTGTCTAAGTGCTAGCTCTGATGATGGTGA |
| | CAAAGAAGTAATAGCAATCAGTGGTTTAGATTAAACCATTTTAGGCATAA |
| | AC[C/T]GTTCTGCTAGAATCCAAGGGGAGATTTTTTCCCATCAAGGAGAC |
| | ATAGCTTGTTGGGAAGATAAGACATACCCAATTGCAGAAGTAATTAATTA |
| | ATTCTTTTTTTTTTTTTTTTTTTTTTTTTTGCGATGGAGTTTCGCTCTTGTT |
| | GCCCAGGCTGGAGTGCAATAGCATGATCTCGGCTCACCACAACCTCTG |
| | CCTCCT |
| RS10935354 | ATAGGCCCTATACAGCTCTCAATTTCTTTAATCAATCTTCCTAGCAGCCC |
| SEQ ID | GTGAGAAATATTACTGTCTTCAGCTTCCTAAAGGAGAAAACAGAGGCCT |
| NO: 8 | GGAGGGATTAAAAGACTTTTCTAAGATTTTAGAGGGCATGTTAGGGTTCA |
| | GGCCCAGGGCTGTCTAACCCAAGGCCTAATTCCTTCTATTACATCCATC |
| | AT[A/G]CATGAGTGAGCACTGGGCATGAGGATACGTCAGTGAAAGGGGC |
| | CCTGTAACATGGACCTTACATTTTGGCTGGGGGAGACAGGCAATGAATA |
| | CATAGGACCATGTTGGGAAGTGCTAAGTACTCTGATGATAACACAGCAG |
| | GGTGAGGTGACAGAGGTCTAGGGAGAGTGGTGTTCAGCAAAAACTTCT |
| | CTGGGGAGAGA |
| RS8095703 | AAAATTTACCAGGTTTAAAAAAAAAAAAACTCAAATGATATTTCAGAAACC |
| SEQ ID | TACCCCTTTCAAAACAAGGAGGAGAAAAATCTTCTCCACAAAAGCACATA |
| NO: 9 | TTGAAAAAATATTTTGGGGGCAAGGCCTGAAAGGGTTGGCAGTGTGCAG |
| | TTCTGTTATTATTCCCGTGGCCATTTTATGGGCCTCAGCAAAACACTGGG |
| | [A/G]TCATTATCTGTCTTCTGGTTACTCCAGGAGAGCTAGCCATCACAAC |
| | CCAATGGAAGAGACTTCAGAGAAACCCACACAGGCACCAGAAGTCCTTC |
| | CCTTTCATCTGCCACTGTGGGGTTTTGTCCTCATCTATTACAATGTTGTC |
| | CAATCTCAGACTGCATTCAGAACAAAGGCTCTCAGACTGAGGATGAGTT |
| | CTTGGA |
| RS10206851 | CCAAATAATTGTTATTGTTGTTTTAACATGGCAATCACGTTATTTGCCATA |
| SEQ ID | TGTGAAAAAGAATATTTAAAATGCTTTTTAAAACTATGTATGTAAAAGAAT |
| NO: 10 | GTTTAAATTGTTTTAAAAATATGTTATATCTACCTTGGCACCATCCTTGCT |
| | GTTGAGAAATGACTTTTACCTGCTTACTTAGAAGGAAATGTCAGAAG[C/T] |
| | AGAAGTACATTTGAATACGATTATTTGAAAGCTTCATCCATTTTTCAAAG |
| | AATGTATACAGTAACACTAAATAGAAAGCATAGTTTATCAACTCTTCACTA |
| | AGAACAGTCTAGCAAGTATATCAGAGTGGCTGTGGTTCCAGTTGGACTA |
| | ACCTAATCATTTATGAAAAGGTGATAATAAGCTTGGACCAAGAGCACCCA |
| RS9542977 | CAGATGTTATGTGAAACTCTGGAGAAATAGTAGCAAGCAAGACTCACAT |
| SEQ ID | GCCTCCTGCCCTCACAGAGCTCCATGATCTGGTGAAAGTGCCAGATATT |
| NO: 11 | TAAACCCATGGATGTGTGCACACAAAATAACAATTCTCTCAAGCGTTGTG |
| | AAGAAAAGTCACAGAGCACTACAAAAGCATGTAAGAGTGAGGCAAAACC |
| | TAT[C/T]GTGTTAGGACAGGGAAGGCTTCTGTGAGCTAACCTGAAGGAT |
| | GAGTAGGGGTGAGCCAGATGAAAAGGCGAGAGAAAAACATTCTGAGCA |
| | GAGACTGCCACTGAGTGCATCCCAGTTTTCCCAACATCTTAACACTGTAT |
| | AATGACTACACTGGATTTTCTTCATCCTGGATCCATGGTTAGACATGTTA |
| | ATATGCCTTC |
| RS4942879 | CCCAGTCTGTGGTATTTTTTTATAGCAGCACAAACAGACTAACACAAGAG |
| SEQ ID | GTGGATAGGATTTGCGAGCATGGACCTTGGAGGTTTGTGGCCTCAATTT |
| NO: 12 | AAAGTGAGTACATTCACCCAGCTGGTGTTTTTCTCTTGCTGCTTGGGCA |
| | CAGAGATGGAGTAAATGGGTCTAATCAAGGATAAAGGGAGAGCCAAAGA |
| | GAT[A/G]GTAATATTTGAAAGGAAGTGTTTTTAATGATGTGCCATGTAATC |
| | TGAGCTGGGTCAGGAATGAAGTGAAAAACTAAGAGATGATGGATGATGA |
| | TAGGGGCTGTGAAAGGAAAACAAATCTTGGGGCCCCCAAATCACTAAGC |
| | TAAAGGAGAAAGTCAAGCTGGGAACTGTTTAGGGCAATCCTGCCTCCCA |
| | TTTTATTCA |
| RS9542954 | TATTACTGCTGAGAAAACTGGGTTTGATAAACTAAAGATGCCCATGTATA |
| SEQ ID | TCAGTCATGCTCCTGGTGAGAACAGGTGGCTCACTGCATAATGAGAGGA |
| NO: 13 | ATATTCAATTAACTATTTACAAAGCTATGGATGACATGTAGGGAAGCCAC |
| | AGAGAGAGTACAGTATCTAGAGCTAGTAAGAGTAGAAGGCCATCACTGT |
| | CC[A/C]CAGGCCTAAAGGAGGTAGAGCAGTCAAAGGAAACAAGAGACAA |
| | GGGAGGCTGCGAGGACAGGGCCACCTGGCAGAGCCATAACCTTAAACT |
| | AGGTAGTCACTTCTTGGCAACTCTGCAGGTAGGGAGCCAACCTCACTTT |
| | TAACCCTCCCTCTGATGCCCAGCTGGTTTACCCCATTGGTGAAAATCAG |
| | TGGGTGAGGGA |
| RS1593478 | CATGAAAAGATACTTAACATTGTAACATCTTTGCATTAGGGAACTGCAAA |
| SEQ ID | TCAAAATCATAACAAAATAGTACTGCATGCTCATTAGGATGACTATAATC |
| NO: 14 | CAAAAGAATAAAAAAGAAAATAACAGGTGTTGGTAGGGATACAGATATAG |
| | AGAAACTGGAGCTCTCATGCCTTGCTGGGGGGCATGTAAAATGATTCTG |
| | C[C/T]GCTTTGGAAAACAGTTTGGTGGTTCCTCAAAAAGTTAAACATATAA |
| | TCCAACAATTCCACCCAAAAGAATTGAAAGCAGGGTCTAGTACACCAAC |
| | GTTCATAGCAGCTTTATTCACATCAAGCCAAAGGTGGAAGCAGCCCAAA |
| | TGTCTACTGATGGATGAGTTGATACACAAAATGTGGTATATATATGCAAT |
| | GGAATA |
| RS9542951 | GCCACTTGAATGCCCCAAAATGGAGAGATGGGCGTGGGAAGAGAAAGA |
| SEQ ID | CACCTCAGCAACACAGAGCTGAGAAAACACTGTGAGTTTTATTTAATTCC |
| NO: 15 | TACTTACCGTTATTTTGCATAGTAAACAAAAGGGATATTTTTGAAAATCCC |
| | TTTGGATAATTTCTGCCACCTAAAATTCTGAGCATTTTGACTCACTGCCTT |
| | [A/G]TAAAAAGAATCAATTAATTGAATAAGAGAAGGGATTCTCCCCTGAT |
| | CTTTTCAAGAATCCTTAAAAGGCACATTTCTCACTAAGGATCTTGAAAGT |
| | GTATTTCTAGCCAATCCCAGGAGTCACTGCTCAGAGATTTACATTTCACA |
| | AATGTAATCAACAGCCTAAGCAGAATATTGACGTTTGGACTGCAGAGCT |
| | CTGCT |
| RS2188534 | AGGGTCCCCAAATATTTCCATTTGAGATGACAAAGTGCTCTTCAGTCATT |
| SEQ ID | TAGCTTACTCTTCAGTTCAGATGACTTATCATCTTGATTTCAGAGAGTTCA |
| NO: 16 | TATATGTCTGTTTTAAAAAACTGGTTCAAAAAGTCTGAAGTTACGAAACTA |
| | AACCAAATATGCATTACTCTCATGTCAAATTACAAGCTCTTAGCTGC[G/T] |
| | GGGATTTTTTCACATGCAGCCTGGAGCCCTTGAAAACCTCTGTTTTCTGT |
| | TAGACTCTCCAGGGTACACAGAAGTTGCCTCATTATTTTAGTTAATGGTG |
| | ACTGCAAATAAGCCCCCCAAGTCATTTAACTATGTGCTTACCACTGCTTT |
| | AAAAGAACCCCAAGTTAGGTCCTCATGTAGGTAAAGGAGCTCCCTTCAC |
| | A |
| RS12524124 | ACTTGGGCCCAAAGGCATTCAACTAGAAAGCTGGTAATAATAACAGCGA |
| SEQ ID | CAGTTTATTGAGTCTTAGTGTTTCTGAGAACTTTTCTAAGTACTTTACACA |
| NO: 17 | TATTAAATTTTTAAATCTTCACATTAGTCCTGTGAGGAAGGTACTATTGTT |
| | ATGTCTGTATTACCCATGGGGATACTGACGCACAAAGAAGTCAAGTAAT |
| | [A/G]TATTTAAGATTCTAGTAAGTGCAGAGCCCAGGTGCATGCAGTGCCT |
| | GGGCTCTGCCACCCATGCAGTGCTGACTAGGGCTTCCACCCATGGATTT |
| | TTTTTTTTTTTTTTTTTTTTTGAGACAGAGTTTCGCTCTTGTTGCCCAGGC |
| | TGGAGTGCAATGGCATGATCTCGGCTCACCACAACCTCCGCCTCTCGG |
| | GTTCAA |
| RS4352629 | CCCATAATAATGAAGGATTGGACCTGATAATCTATCAGGTACATTTTAGC |
| SEQ ID | CTGAAATTTATTTGTACACACGCACAAACACAGACATGTGCACACACACA |
| NO: 18 | TACACATATATATATAACATTTATAAATTTTAAAACATAAAGCTATACTAGA |
| | AATGAAAGCTTATATATTGAACTGCCCCACCTTTCTATTTGCAGCCAG[C/ |
| | T]TACCACCCCAGTCTAATGTTTCACTTTATATAAATTCATTTATTCTTTTA |
| | CTCATTTCAAATATATGATGATGTAACTATAAAATCAACATTTAGTCACTC |
| | TGAATAACCCAAAATAGCAAATAATTTAAAAATCACTTCCACTTGACTTTA |
| | GAATCTATTACATGCATTGTTTTTCCAGAAAATTTACCTCATAATTAT |
| RS7448716 | CTACTTATATGATTAGAGAACAAGAATACTAGGGGGAAAATCAGCATGCA |
| SEQ ID | TATAATCTAAGAAATTGTCATTATAATTTTAAAATCCTTTGCAAAATCAGTA |
| NO: 19 | AATATGAGTTTAACTTATATAATGATACACACACACACTGATATGATGCTT |
| | TATTGTCTAAACACTGGCTGCTTGTGGAGACGTATTCTGGTAACAAA[A/G] |
| | AATATAGCATCTTAAAATTGATGCTAGCATTGTATATCCAAATAGAGAGT |
| | AAATGCAACCAGAATATTTTTTATATGTTTAACATTGTAGTGTTGCTGACA |
| | TCATTATATATTTGGTTATGTTAATCTCAAAATGCACAATATAGCTGTATG |
| | ATCTGTATAATGCAAAAAAATGTAGAGCTTCATTTTGATATTTATTAT |
| RS11873533 | CTGGAAGGGAACAATGGAAGAGGTGCATTAGTCACATTCCAAAATGCAG |
| SEQ ID | GAAGCAATAACATGTGGCACTATTGTCATTTATGTAGCACCCTAAATACT |
| NO: 20 | GGGACAAATGACATAGATGCCCTTCTGTGATTACTAAACTCCCCCACAG |
| | TGTCTCAGAAGGAAGAGCTTTTGACAGGAAATCATCAAGATCTGATGAC |
| | ATT[A/C]GAGAGCAATTAACATTCTCTTCAACCATGAACTAATTGCCTCAT |
| | TCACATTTTTCTAGCCATCCTAGGAAGCAGATAATAAGCAGCAATTGTCC |
| | TGCCCAGGAATTCTGACTTGTGTAATTTGTAAAGCTTTTCTTTGTATCTAT |
| | TTCTTTCCTGTGGCCATCTTTTTGTTTTTGGACTGTTTGGTAACAGTAAGT |
| | GGGT |
| RS10062658 | ATCATTCAGTATTAAGAGAGAAATGAATACATTTTCAGATATACAAGAATT |
| SEQ ID | CAGTTTACCTCCCACAGAATCTCTGAAAAAAATATTAGATTACTATAGTTA |
| NO: 21 | AAAAGGAAAATAAAATAAGTCCTGTTAGAAATAATTGGTAAAAAAGCAAA |
| | GGTGATGAAAACTTATTGAAATATATTATTAAAGTAATTGTTAAAAAT[A/G] |
| | TACACTAAATCTAGAATATATAAATGTAGCAGTTGTTAAGGGGAAGGGGA |
| | AATAGAAGTGGAAGAAAATGAACATTAGAACAAATGTTTAGCAGTGGGAT |
| | TATTTTATTGGAAGTCTAATGTAAGAAGTATATTCTCCAGGGAGGTATTT |
| | CAAGGACATATGAATAGTAAAGGGATAATAAAACAACTCTATAAGGTAGT |
| RS12547917 | CACACACAACGCTGGGCCCAGTAAATAAGTTTTGTTTTTTCCCAGGGAA |
| SEQ ID | AAGTTGAACAACAATGGTGAGACCAGGAAGGCTCTCCGTTCACAGGAAA |
| NO: 22 | TACTGTGTCACCGCTCGGCCGCAGGCTGTGTGAGGTCACGGGCGACGC |
| | TCGGGTCACGTGTGGCGGCTCCTGTTCACAGTGCCGTGTGTGATAAACT |
| | GGGAC[C/T]TTCTGGTGAGGGGAGACTGGCGGGGGGTGGGGAGGGCA |
| | AGGAGTGGGAAAGTCGCCTATAAATGTTTAACAAAAGATCCGCAATGGG |
| | AACAGGAACTTGCATTCTTTCTTTCAATGGACAAAGCTTCCACATCAAGA |
| | TACGCTTGTGTGCTGGGACCAAATGCCACAGTGCGGCGAAACTCGTGA |
| | GCACAAGTCCTGCGT |
| RS1038268 | ACAACAGGGTATCCTAGCCCAGCAAAATTGACTCATAAATTTAATGATCA |
| SEQ ID | CGCAATTGGTAATTCTAAATCCAGTCAGAAGTCTACATTCTGTGTCCACA |
| NO: 23 | GTGTCATGTCTAGATGTTGGTCCAGTCTCCCATGGACTGTGCCTTGTTAT |
| | TTGTTTTCTCTTTGCTAAGCCACATCCCCTGAGGGCTCTGTTTATGCTCA |
| | [C/T]TGCAAAATCTTTGACTTTTTAACTTACTGGGCATATTGTCTTCCTACT |
| | TTTGTTCTCTTCTGTTATTTTATTTACTTGACTCTGACATGTCTCATTCCC |
| RS2375811 | TTTCAATGGGACTGGTTGGACAGTGGGTGCAGCCCATGAAGGGCAAGC |
| SEQ ID | CAAAGCAGGCCGGGGCATCACCTCACCCGGGAAGCACAAGGGGTCAG |
| NO: 24 | GCGATTTCTCTTTCCTAGTCAAGGGAAGCCATGGCAGACTGCACCTGGA |
| | AAAACGAGACACTTCCACCCAAATACTGCGTTTTTCGCAAGGTCTTAGCA |
| | ACTAAC[A/G]GACAAGGAGATTCTCTCCCGTGCCTGGCTCGGCTGGTCC |
| | CACACCCACGGTGACTTGTTCACTGCTAGCACAGCAGTTTGAGATCGAA |
| | CTACGAGGCAACAACCTGGCTAAGGGAGGGGCATCTGCCATTGCTGAG |
| | GCTTGAGTAGGTAAACAAAGTGGCCAGGAAGCTCGAACTGGGTGGAGC |
| | CCACTGCAGCTTAGCA |
| RS1352671 | ACTTTAGGGACTTTGAGTGATGGACAACCCCCTATCAGATATCATCAGC |
| SEQ ID | CTGAAACATCCTTATCTTGGCATTAAATTAGAAGGAACCCCAGACCCTGC |
| NO: 25 | GTACCAGAATTGTTAGAATCACAGTCTCAGTAAAGAACCAACTCCTGATC |
| | ACTTCTCTAAAGGAAAGTTCTAGAAGTCTGCACACTCTGCAGTCACTTTC |
| | A[A/C]TTCTATCCAAGTGTACACTTAGAACTCTAGAAAACACTACGGAGA |
| | GTCTTCAGCCAGGTAAAGCCTAAAACCAGCAAAGAACAGGGAGAGTGA |
| | GGGA |
| RS364331 | CGGATTATCACAGTTCTCAAAAGAGGAGTATGCATTTGCTTGCTCCAATT |
| SEQ ID | CCTCTTCTTCTACACTCTCTTAAGCATTCCTCAACCAGTCTAATATCTCAT |
| NO: 26 | AGTTCCCCAAAACTGCTCTGTTCAAGACCATTAGTAAGATCTTTGATGTT |
| | AATCTGTGGACCGTATCTCTGTCCTTATTTTACTTGAAGCCCAACAGCA |
| | [A/C]ATAAAAAAGTTGTTCTCCTCTCCTCCCTGCTACACTTTCCTTATGTG |
| | GCTTGCTGGGCTCCTCAGTCCCCTGTGAAAAACTCTGACATGGAGATAC |
| | TGCAGACCAGTAGAAGGGCTGGGCAGACACTATACAGAAACAGTATGC |
| | CCTACATGCTCCTTGGCTAAATCTCTAGAATTTTTTTCAGAACTCATCCA |
| | CAAATT |
| RS1924949 | ATTTTATCTCATTTACTTATTAAATCAAACCAATATTTTATGAAGTGATTCC |
| SEQ ID | AGTATTGGAATAAAAATGTAATTCTTTAATCATTAAAAAATCTTTATGAATA |
| NO: 27 | CCTTACATCAACTGTAGGGGACCAACCAGGGAAAAGCAGGGAGACTTG |
| | TAGAATCTACACCTCCAGAACAACCGACCTCCATCTTCTGGACAACTC[A/ |
| | G]TCTTCTAAAGTGCAGGACAGACTAGTTGGGGGAGAAAGGAGGAAATG |
| | AAAGAGATAGACTAAAAGGGAGGGAGAGAACAGATATTTTTTAAGTACC |
| | TGTTATGTTCTGGATACAGCACAGAGTACATTGTATCTATTATTATAAGG |
| | CATAAAGAAAGATTTCTCAGGTTTTTGGAGTCAGATTGCAATATAAAATA |
| | ATAG |
| RS11025990 | TAACTTCAAATTGTTTTGCAAAATCTCTGTCATAAAAATGCTTACCAACAA |
| SEQ ID | ATACTGATACTAAATTTAGATGTGGGGGTATTAGTTATAATCCTGAAGTG |
| NO: 28 | GGAGGGGGAACTTCTTAATTCCAATTTAGTTCTAAGAGAAGGAAGAGTA |
| | TTTAGGCCCAGAGAAGGTTACGCTTAAAGGTCTGATAGTGTTTTCTTTGA |
| | [A/G]AAATATGTCTCAAACTAGAGAATAAAACTAATTATCTCATCTAAGTT |
| | ACCTAGAGACATTTATGCTCATCAGTTTGATAAAGGACTGCAAGTAGACA |
| | CAGAAGCTGTATTTTCAGTCTTGAACCCAGCAATAGTACATTAACAAGAT |
| | TGGGGCAAGGCAAAGGGACTTTTGTGGCACAAGATACAATATATGGATT |
| | GCGT |
| RS3758562 | CTCTTCAAAGGCCTTTGCCCTTGGGTACCACAGGTTCTGAGACAAGAGG |
| SEQ ID | GCTATGGAGAGCCCCCATTATAGCTGGAGCCTCCTGCCCTGCCCAAAG |
| NO: 29 | GTGTGACTTGAAGGGTGGAATTTCAGGCAGCGTGGCTCGCCCCAGGGA |
| | GGCAAAGAGGCCAGGGGAATCTTCAAAGGCCCTGGGCTCATCCCAGCT |
| | AGGAGGC[A/G]GGCACAGTCATAACCCTAATCCAGTGAACTCAGCCCTC |
| | ATCCTGACTCTCATGGTATTCTGTCCCAGGGAGCCTCTTTCCAGCTTTCT |
| | TAGAAGCTTTAATGTCAGCACTTGCAGGGCCTTAGAAACTGCACGCTAC |
| | CTCTTCATTTCATACATGAGGAAACTGAGGCCCAGGGTGGACACAGGGC |
| | TGCCCAGCGAGTTA |
| RS10156056 | ATTATAAAGCAAAGCACTAACCTCATAGAATACCTGAGTCAAGTTCCCTG |
| SEQ ID | TGTTCTCATTTTCTAGCCTCTTCTACCAGACACTATGAAAAATAACAGCC |
| NO: 30 | CCATCTCTCCAGAAAATCTTAGGAGATATAGGCGTGCTGAATTTAAGGT |
| | GTCTGTGGCACATGCAAGTGGATCAGCCACTGGGCTGTCCAGAATGCA |
| | AGA[C/G]AGAACTCAGAGTTGGGGACATAAACTTGGCAGTCATCTGTGTA |
| | AAAGAGAAAAGGTAGGTAAAGTCCCACAAGGATGGGTTGGCCTACAGAA |
| | GGCACAGAGAGAAGAGAGCCTGGTTTAGCATGGGCTACGATCAGAGTC |
| | CCTGGGTTCAAATCTTGGCTCCACCCATTTCTATCTGGTTGCTCTAGGG |
| | CATGTTACCTA |

Polymorphisms in linkage disequilibrium with a SNP of table 1 can be identified by by methods known in the art. For example, Develin and Risch (Genomics, 1995) provide guidance for determining the parameter delta (also referred to as the “r”) as a standard measure of the disequilibrium. Gabriel et al. (Science, 2002) provides instructions for finding the maximal r² value in populations for disease gene mapping. Further, Carlson et al. (Am. J. Hum. Genet. (2003) disclose methods for selecting and analyzing polymorphisms based on linkage disequilibrium for disease gene association mapping. Stoyanovich and Pe'er (Bioinformatics, 2008) show that polymorphisms in linkage disequilibrium with indentified SNPs have virtually identical response profiles. Currently, several databases provide datasets that can be searched for polymorphisms in strong linkage disequilibrium, which can be accessed by the following addresses: http://1000.genomes.org, http://www.hapmap.org, http://www.broadinsitute.org/mpg/snap. An example workflow for determining SNPs linkage disequilibrium to a specific SNP is outlined in Uhr et al. (Neuron, 2008).

SNP in strong linkage disequilibrium as used herein means that the SNP is in linkage disequilibrium with an r² higher than 0.7 or higher than 0.8 in the tested population or an ethnically close reference population with the identified SNP.

In another embodiment, at least 20, optionally at least 25 or at least 30 SNPs selected from the group of table 1 are determined in step (a) and integrated by the prediction algorithm in step (b). For example, all intergenic SNPs of table 1 are determined in step (a) and integrated by the prediction algorithm in step (b). In another exemplary embodiment, all SNPs selected from the group of table 1 are determined in step (a) and integrated by the prediction algorithm of step (b).

Typically, in step (a) of the method of predicting a treatment response to CRHR1 antagonists, the presence or absence of those SNPs is determined which were identified to be associated with values indicative for normal CRH activity or CRH overactivity and were thus considered when determining the prediction algorithm by machine-learning as described above.

Another aspect of the invention concerns biomarkers. The biomarker or the set of biomarkers may be selected from one or more biomarkers of the group comprising comprising:
SNP rs6437726,
SNP rs1986684,
SNP rs7380830,
SNP rs3903768,
SNP rs7325978,
SNP rs13585,
SNP rs9368373,
SNP rs10935354,
SNP rs8095703,
SNP rs10206851,
SNP rs9542977,
SNP rs4942879,
SNP rs9542954,
SNP rs1593478,
SNP rs9542951,
SNP rs2188534,
SNP rs12524124,
SNP rs4352629,
SNP rs7448716,
SNP rs11873533,
SNP rs10062658,
SNP rs12547917,
SNP rs1038268,
SNP rs2375811,
SNP rs1352671,
SNP rs364331,
SNP rs1924949,
SNP rs11025990,
SNP rs3758562, and/or
SNP rs10156056.

In one embodiment, the biomarker or the set of biomarkers is selected from one or more biomarkers as described above, wherein
SNP rs6437726 is represented by a single polymorphic change at position 201 of SEQ ID NO: 1, wherein in one or two alleles the wild-type nucleotide A is replaced by indicator nucleotide G,
SNP rs1986684 is represented by a single polymorphic change at position 201 of SEQ ID NO: 2, wherein in one or two alleles the wild-type nucleotide A is replaced by indicator nucleotide G,
SNP rs7380830 is represented by a single polymorphic change at position 201 of SEQ ID NO: 3, wherein in one or two alleles the wild-type nucleotide C is replaced by indicator nucleotide T,
SNP rs3903768 is represented by a single polymorphic change at position 201 of SEQ ID NO: 4, wherein in one or two alleles the wild-type nucleotide A is replaced by indicator nucleotide G,
SNP rs7325978 is represented by a single polymorphic change at position 201 of SEQ ID NO: 5, wherein in one or two alleles the wild-type nucleotide C is replaced by indicator nucleotide T,
SNP rs13585 is represented by a single polymorphic change at position 185 of SEQ ID NO: 6, wherein in one or two alleles the wild-type nucleotide C is replaced by indicator nucleotide T,
SNP rs9368373 is represented by a single polymorphic change at position 201 of SEQ ID NO: 7, wherein in one or two alleles the wild-type nucleotide C is replaced by indicator nucleotide T,
SNP rs10935354 which is represented by a single polymorphic change at position 201 of SEQ ID NO: 8, wherein in one or two alleles the wild-type nucleotide A is replaced by indicator nucleotide G,
SNP rs8095703 is represented by a single polymorphic change at position 201 of SEQ ID NO: 9, wherein in one or two alleles the wild-type nucleotide A is replaced by indicator nucleotide G,
SNP rs10206851 is represented by a single polymorphic change at position 201 of SEQ ID NO: 10, wherein in one or two alleles the wild-type nucleotide C is replaced by indicator nucleotide T,
SNP rs9542977 is represented by a single polymorphic change at position 201 of SEQ ID NO: 11, wherein in one or two alleles the wild-type nucleotide C is replaced by indicator nucleotide T,
SNP rs4942879 is represented by a single polymorphic change at position 201 of SEQ ID NO: 12, wherein in one or two alleles the wild-type nucleotide A is replaced by indicator nucleotide G,
SNP rs9542954 is represented by a single polymorphic change at position 201 of SEQ ID NO: 13, wherein in one or two alleles the wild-type nucleotide A is replaced by indicator nucleotide C,
SNP rs1593478 is represented by a single polymorphic change at position 201 of SEQ ID NO: 14, wherein in one or two alleles the wild-type nucleotide C is replaced by indicator nucleotide T,
SNP rs9542951 is represented by a single polymorphic change at position 201 of SEQ ID NO: 15, wherein in one or two alleles the wild-type nucleotide A is replaced by indicator nucleotide G,
SNP rs2188534 is represented by a single polymorphic change at position 200 of SEQ ID NO: 16, wherein in one or two alleles the wild-type nucleotide G is replaced by indicator nucleotide T,
SNP rs12524124 is represented by a single polymorphic change at position 201 of SEQ ID NO: 17, wherein in one or two alleles the wild-type nucleotide A is replaced by indicator nucleotide G,
SNP rs4352629 is represented by a single polymorphic change at position 201 of SEQ ID NO: 18, wherein in one or two alleles the wild-type nucleotide C is replaced by indicator nucleotide T,
SNP rs7448716 is represented by a single polymorphic change at position 201 of SEQ ID NO: 19, wherein in one or two alleles the wild-type nucleotide A is replaced by indicator nucleotide G,
SNP rs11873533 is represented by a single polymorphic change at position 201 of SEQ ID NO: 20, wherein in one or two alleles the wild-type nucleotide A is replaced by indicator nucleotide C,
SNP rs10062658 is represented by a single polymorphic change at position 201 of SEQ ID NO: 21, wherein in one or two alleles the wild-type nucleotide A is replaced by indicator nucleotide G,
SNP rs12547917 is represented by a single polymorphic change at position 201 of SEQ ID NO: 22, wherein in one or two alleles the wild-type nucleotide C is replaced by indicator nucleotide T,
SNP rs1038268 is represented by a single polymorphic change at position 201 of SEQ ID NO: 23, wherein in one or two alleles the wild-type nucleotide C is replaced by indicator nucleotide T,
SNP rs2375811 is represented by a single polymorphic change at position 201 of SEQ ID NO: 24, wherein in one or two alleles the wild-type nucleotide A is replaced by indicator nucleotide G,
SNP rs1352671 is represented by a single polymorphic change at position 201 of SEQ ID NO: 25, wherein in one or two alleles the wild-type nucleotide A is replaced by indicator nucleotide C,
SNP rs364331 is represented by a single polymorphic change at position 201 of SEQ ID NO: 26, wherein in one or two alleles the wild-type nucleotide A is replaced by indicator nucleotide C,
SNP rs1924949 is represented by a single polymorphic change at position 201 of SEQ ID NO: 27, wherein in one or two alleles the wild-type nucleotide A is replaced by indicator nucleotide G,
SNP rs11025990 is represented by a single polymorphic change at position 201 of SEQ ID NO: 28, wherein in one or two alleles the wild-type nucleotide A is replaced by indicator nucleotide G,
SNP rs3758562 is represented by a single polymorphic change at position 201 of SEQ ID NO: 29, wherein in one or two alleles the wild-type nucleotide A is replaced by indicator nucleotide G, and/or
SNP rs10156056 is represented by a single polymorphic change at position 201 of SEQ ID NO: 30, wherein in one or two alleles the wild-type nucleotide C is replaced by indicator nucleotide G.

In a specific embodiment, a set of biomarkers or a group of biomarkers comprises at least 15, at least 20, at least 25 or all of the following biomarkers:
SNP rs6437726,
SNP rs1986684,
SNP rs7380830,
SNP rs3903768,
SNP rs7325978,
SNP rs13585,
SNP rs9368373,
SNP rs10935354,
SNP rs8095703,
SNP rs10206851,
SNP rs9542977,
SNP rs4942879,
SNP rs9542954,
SNP rs1593478,
SNP rs9542951,
SNP rs2188534,
SNP rs12524124,
SNP rs4352629,
SNP rs7448716,
SNP rs11873533,
SNP rs10062658,
SNP rs12547917,
SNP rs1038268,
SNP rs2375811,
SNP rs1352671,
SNP rs364331,
SNP rs1924949,
SNP rs11025990,
SNP rs3758562,
SNP rs10156056.

In another specific embodiment, a set of biomarkers or a group of biomarkers comprises at least 15, at least 20, at least 25 or all of the following biomarkers:
SNP rs6437726 which is represented by a single polymorphic change at position 201 of SEQ ID NO: 1, wherein in one or two alleles the wild-type nucleotide A is replaced by indicator nucleotide G,
SNP rs1986684 which is represented by a single polymorphic change at position 201 of SEQ ID NO: 2, wherein in one or two alleles the wild-type nucleotide A is replaced by indicator nucleotide G,
SNP rs7380830 which is represented by a single polymorphic change at position 201 of SEQ ID NO: 3, wherein in one or two alleles the wild-type nucleotide C is replaced by indicator nucleotide T,
SNP rs3903768 which is represented by a single polymorphic change at position 201 of SEQ ID NO: 4, wherein in one or two alleles the wild-type nucleotide A is replaced by indicator nucleotide G,
SNP rs7325978 which is represented by a single polymorphic change at position 201 of SEQ ID NO: 5, wherein in one or two alleles the wild-type nucleotide C is replaced by indicator nucleotide T,
SNP rs13585 which is represented by a single polymorphic change at position 185 of SEQ ID NO: 6, wherein in one or two alleles the wild-type nucleotide C is replaced by indicator nucleotide T,
SNP rs9368373 which is represented by a single polymorphic change at position 201 of SEQ ID NO: 7, wherein in one or two alleles the wild-type nucleotide C is replaced by indicator nucleotide T,
SNP rs10935354 which is represented by a single polymorphic change at position 201 of SEQ ID NO: 8, wherein in one or two alleles the wild-type nucleotide A is replaced by indicator nucleotide G,
SNP rs8095703 which is represented by a single polymorphic change at position 201 of SEQ ID NO: 9, wherein in one or two alleles the wild-type nucleotide A is replaced by indicator nucleotide G,
SNP rs10206851 which is represented by a single polymorphic change at position 201 of SEQ ID NO: 10, wherein in one or two alleles the wild-type nucleotide C is replaced by indicator nucleotide T,
SNP rs9542977 which is represented by a single polymorphic change at position 201 of SEQ ID NO: 11, wherein in one or two alleles the wild-type nucleotide C is replaced by indicator nucleotide T,
SNP rs4942879 which is represented by a single polymorphic change at position 201 of SEQ ID NO: 12, wherein in one or two alleles the wild-type nucleotide A is replaced by indicator nucleotide G,
SNP rs9542954 which is represented by a single polymorphic change at position 201 of SEQ ID NO: 13, wherein in one or two alleles the wild-type nucleotide A is replaced by indicator nucleotide C,
SNP rs1593478 which is represented by a single polymorphic change at position 201 of SEQ ID NO: 14, wherein in one or two alleles the wild-type nucleotide C is replaced by indicator nucleotide T,
SNP rs9542951 which is represented by a single polymorphic change at position 201 of SEQ ID NO: 15, wherein in one or two alleles the wild-type nucleotide A is replaced by indicator nucleotide G,
SNP rs2188534 which is represented by a single polymorphic change at position 200 of SEQ ID NO: 16, wherein in one or two alleles the wild-type nucleotide G is replaced by indicator nucleotide T,
SNP rs12524124 which is represented by a single polymorphic change at position 201 of SEQ ID NO: 17, wherein in one or two alleles the wild-type nucleotide A is replaced by indicator nucleotide G,
SNP rs4352629 which is represented by a single polymorphic change at position 201 of SEQ ID NO: 18, wherein in one or two alleles the wild-type nucleotide C is replaced by indicator nucleotide T,
SNP rs7448716 which is represented by a single polymorphic change at position 201 of SEQ ID NO: 19, wherein in one or two alleles the wild-type nucleotide A is replaced by indicator nucleotide G,
SNP rs11873533 which is represented by a single polymorphic change at position 201 of SEQ ID NO: 20, wherein in one or two alleles the wild-type nucleotide A is replaced by indicator nucleotide C,
SNP rs10062658 which is represented by a single polymorphic change at position 201 of SEQ ID NO: 21, wherein in one or two alleles the wild-type nucleotide A is replaced by indicator nucleotide G,
SNP rs12547917 which is represented by a single polymorphic change at position 201 of SEQ ID NO: 22, wherein in one or two alleles the wild-type nucleotide C is replaced by indicator nucleotide T,
SNP rs1038268 which is represented by a single polymorphic change at position 201 of SEQ ID NO: 23, wherein in one or two alleles the wild-type nucleotide C is replaced by indicator nucleotide T,
SNP rs2375811 which is represented by a single polymorphic change at position 201 of SEQ ID NO: 24, wherein in one or two alleles the wild-type nucleotide A is replaced by indicator nucleotide G,
SNP rs1352671 which is represented by a single polymorphic change at position 201 of SEQ ID NO: 25, wherein in one or two alleles the wild-type nucleotide A is replaced by indicator nucleotide C,
SNP rs364331 which is represented by a single polymorphic change at position 201 of SEQ ID NO: 26, wherein in one or two alleles the wild-type nucleotide A is replaced by indicator nucleotide C,
SNP rs1924949 which is represented by a single polymorphic change at position 201 of SEQ ID NO: 27, wherein in one or two alleles the wild-type nucleotide A is replaced by indicator nucleotide G,
SNP rs11025990 which is represented by a single polymorphic change at position 201 of SEQ ID NO: 28, wherein in one or two alleles the wild-type nucleotide A is replaced by indicator nucleotide G,
SNP rs3758562 which is represented by a single polymorphic change at position 201 of SEQ ID NO: 29, wherein in one or two alleles the wild-type nucleotide A is replaced by indicator nucleotide G,
SNP rs10156056 which is represented by a single polymorphic change at position 201 of SEQ ID NO: 30, wherein in one or two alleles the wild-type nucleotide C is replaced by indicator nucleotide G.

For example, the set of biomarkers or the group of biomarkers consists of the following biomarkers:
SNP rs6437726,
SNP rs1986684,
SNP rs7380830,
SNP rs3903768,
SNP rs7325978,
SNP rs13585,
SNP rs9368373,
SNP rs10935354,
SNP rs8095703,
SNP rs10206851,
SNP rs9542977,
SNP rs4942879,
SNP rs9542954,
SNP rs1593478,
SNP rs9542951,
SNP rs2188534,
SNP rs12524124,
SNP rs4352629,
SNP rs7448716,
SNP rs11873533,
SNP rs10062658,
SNP rs12547917,
SNP rs1038268,
SNP rs2375811,
SNP rs1352671,
SNP rs364331,
SNP rs1924949,
SNP rs11025990,
SNP rs3758562,
SNP rs10156056.

In another example, the set of biomarkers or the group of biomarkers consists of the following biomarkers:
SNP rs6437726 which is represented by a single polymorphic change at position 201 of SEQ ID NO: 1, wherein in one or two alleles the wild-type nucleotide A is replaced by indicator nucleotide G,
SNP rs1986684 which is represented by a single polymorphic change at position 201 of SEQ ID NO: 2, wherein in one or two alleles the wild-type nucleotide A is replaced by indicator nucleotide G,
SNP rs7380830 which is represented by a single polymorphic change at position 201 of SEQ ID NO: 3, wherein in one or two alleles the wild-type nucleotide C is replaced by indicator nucleotide T,
SNP rs3903768 which is represented by a single polymorphic change at position 201 of SEQ ID NO: 4, wherein in one or two alleles the wild-type nucleotide A is replaced by indicator nucleotide G,
SNP rs7325978 which is represented by a single polymorphic change at position 201 of SEQ ID NO: 5, wherein in one or two alleles the wild-type nucleotide C is replaced by indicator nucleotide T,
SNP rs13585 which is represented by a single polymorphic change at position 185 of SEQ ID NO: 6, wherein in one or two alleles the wild-type nucleotide C is replaced by indicator nucleotide T,
SNP rs9368373 which is represented by a single polymorphic change at position 201 of SEQ ID NO: 7, wherein in one or two alleles the wild-type nucleotide C is replaced by indicator nucleotide T,
SNP rs10935354 which is represented by a single polymorphic change at position 201 of SEQ ID NO: 8, wherein in one or two alleles the wild-type nucleotide A is replaced by indicator nucleotide G,
SNP rs8095703 which is represented by a single polymorphic change at position 201 of SEQ ID NO: 9, wherein in one or two alleles the wild-type nucleotide A is replaced by indicator nucleotide G,
SNP rs10206851 which is represented by a single polymorphic change at position 201 of SEQ ID NO: 10, wherein in one or two alleles the wild-type nucleotide C is replaced by indicator nucleotide T,
SNP rs9542977 which is represented by a single polymorphic change at position 201 of SEQ ID NO: 11, wherein in one or two alleles the wild-type nucleotide C is replaced by indicator nucleotide T,
SNP rs4942879 which is represented by a single polymorphic change at position 201 of SEQ ID NO: 12, wherein in one or two alleles the wild-type nucleotide A is replaced by indicator nucleotide G,
SNP rs9542954 which is represented by a single polymorphic change at position 201 of SEQ ID NO: 13, wherein in one or two alleles the wild-type nucleotide A is replaced by indicator nucleotide C,
SNP rs1593478 which is represented by a single polymorphic change at position 201 of SEQ ID NO: 14, wherein in one or two alleles the wild-type nucleotide C is replaced by indicator nucleotide T,
SNP rs9542951 which is represented by a single polymorphic change at position 201 of SEQ ID NO: 15, wherein in one or two alleles the wild-type nucleotide A is replaced by indicator nucleotide G,
SNP rs2188534 which is represented by a single polymorphic change at position 200 of SEQ ID NO: 16, wherein in one or two alleles the wild-type nucleotide G is replaced by indicator nucleotide T,
SNP rs12524124 which is represented by a single polymorphic change at position 201 of SEQ ID NO: 17, wherein in one or two alleles the wild-type nucleotide A is replaced by indicator nucleotide G,
SNP rs4352629 which is represented by a single polymorphic change at position 201 of SEQ ID NO: 18, wherein in one or two alleles the wild-type nucleotide C is replaced by indicator nucleotide T,
SNP rs7448716 which is represented by a single polymorphic change at position 201 of SEQ ID NO: 19, wherein in one or two alleles the wild-type nucleotide A is replaced by indicator nucleotide G,
SNP rs11873533 which is represented by a single polymorphic change at position 201 of SEQ ID NO: 20, wherein in one or two alleles the wild-type nucleotide A is replaced by indicator nucleotide C,
SNP rs10062658 which is represented by a single polymorphic change at position 201 of SEQ ID NO: 21, wherein in one or two alleles the wild-type nucleotide A is replaced by indicator nucleotide G,
SNP rs12547917 which is represented by a single polymorphic change at position 201 of SEQ ID NO: 22, wherein in one or two alleles the wild-type nucleotide C is replaced by indicator nucleotide T,
SNP rs1038268 which is represented by a single polymorphic change at position 201 of SEQ ID NO: 23, wherein in one or two alleles the wild-type nucleotide C is replaced by indicator nucleotide T,
SNP rs2375811 which is represented by a single polymorphic change at position 201 of SEQ ID NO: 24, wherein in one or two alleles the wild-type nucleotide A is replaced by indicator nucleotide G,
SNP rs1352671 which is represented by a single polymorphic change at position 201 of SEQ ID NO: 25, wherein in one or two alleles the wild-type nucleotide A is replaced by indicator nucleotide C,
SNP rs364331 which is represented by a single polymorphic change at position 201 of SEQ ID NO: 26, wherein in one or two alleles the wild-type nucleotide A is replaced by indicator nucleotide C,
SNP rs1924949 which is represented by a single polymorphic change at position 201 of SEQ ID NO: 27, wherein in one or two alleles the wild-type nucleotide A is replaced by indicator nucleotide G,
SNP rs11025990 which is represented by a single polymorphic change at position 201 of SEQ ID NO: 28, wherein in one or two alleles the wild-type nucleotide A is replaced by indicator nucleotide G,
SNP rs3758562 which is represented by a single polymorphic change at position 201 of SEQ ID NO: 29, wherein in one or two alleles the wild-type nucleotide A is replaced by indicator nucleotide G,
SNP rs10156056 which is represented by a single polymorphic change at position 201 of SEQ ID NO: 30, wherein in one or two alleles the wild-type nucleotide C is replaced by indicator nucleotide G.

The biomarkers or the group of biomarkers as described above may constitute markers for the treatment response to CRHR1 antagonists in patients with depressive and/or anxiety symptoms. In particular, the above defined biomarkers or groups of biomarkers are suitable to predict the treatment response to a CRHR1 antagonist in a patient with depressive and/or anxiety symptoms

Further, the group of biomarkers may additionally comprise REM density.

Another aspect of the invention concerns a method for detecting CRH overactivity in a patient with depressive symptoms and/or anxiety symptoms, comprising determining the status of a biomarker or a group of biomarkers as defined above in a nucleic acid isolated from a patient's sample, wherein the presence of indicator nucleotides as defined above is indicative for CRH overactivity.

In some embodiments of the method for detecting CRH overactivity in a patient with depressive symptoms and/or anxiety symptoms, the status of at least 15, at least 20, at least 25 or all of the biomarkers as defined above is determined in a nucleic acid isolated from a patient's sample.

Another aspect of the invention concerns a method for monitoring depression and/or anxiety therapy of a patient with a CRHR1 antagonist comprising the step of determining the status of a biomarker or a group of biomarkers as defined above before and during the therapy, optionally also after the therapy.

In some embodiments of the method for monitoring depression and/or anxiety therapy of a patient with a CRHR1 antagonist, the status of at least 15, at least 20, at least 25 or all of the biomarkers as defined above is determined in a nucleic acid isolated from a patient's sample.

The term “monitoring” as used herein relates to the accompaniment of a depression and/or anxiety therapy during a certain period of time, typically during 6 months, 1 year, 2 years, 3 years, 5 years, 10 years, or any other period of time. The term “accompaniment” means that states of disease as defined herein and, in particular, changes of these states of disease may be detected by comparing the status of a biomarker of the present invention in a sample in any type of a periodical time segment, e.g. every week, every 2 weeks, every month, every 2, 3, 4, 5, 6, 7, 8, 9, 10, 11 or 12 month, every 1.5 year, every 2, 3, 4, 5, 6, 7, 8, 9 or 10 years, during any period of time, e.g. during 2 weeks, 3 weeks, 1, 2, 3, 4, 5, 6, 7, 8, 9, 10, 11, 12 months, 1.5, 2, 3, 4, 5, 6, 7, 8, 9, 10, 15 or 20 years, respectively. The term “before therapy of a patient with a CRHR1 antagonist” as used herein means that a patient or patient's sample may analyzed after an initial diagnosis of depression and/or anxiety and before the commencement of a treatment with a CRHR1 antagonist. The corresponding period of time may be 1 hour, 12 hours, 1 day, 2 days, 3 days, 4 days, 5 days, 6 days, 1 week, 2 weeks, 3 weeks, 4 weeks, 2 months, 3 months, 4 months, 5 months, 6 months, or more or any period of time in between these values. The term “during therapy of a patient with a CRHR1 antagonist” as used refers to the determination during the entire or during a part of a therapeutic treatment. For instance, the determination may be carried out between administration steps, or at a defined interval of 1 hour, 12 hours, 1 day, 2 days, 3 days, 4 days, 5 days, 6 days, 1 week, 2 weeks, 3 weeks, 4 weeks, 2 months, 3 months, 4 months, 5 months, 6 months, or more or any period of time in between these values. In a specific embodiment, the monitoring may also be carried out after the therapy of a patient with a CRHR1 antagonist, e.g. 1 hour, 12 hours, 1 day, 2 days, 3 days, 4 days, 5 days, 6 days, 1 week, 2 weeks, 3 weeks, 4 weeks, 2 months, 3 months, 4 months, 5 months, 6 months, or more or any period of time in between these values after the termination of the therapy of a patient with a CRHR1 antagonist. Changes of the status of biomarkers as defined herein above may provide the medical professional with indications regarding CRH overactivity and may lead to a modification of administration, the inclusion of other or more or less medicaments, a combination with further medicaments or any other suitable decision to increase the health of a patient.

Another aspect of the invention concerns a method of identifying a patient with depressive symptoms and/or anxiety symptoms as eligible for a therapy with a CRHR1 antagonist, comprising:

(a) determining in a nucleic acid sample isolated from a patient's sample the status of a biomarker as defined above;

(b) identifying the patient as eligible for a therapy with a CRHR1 antagonist, where the algorithm provided by the method of claim 1 predicts that patient responds to the treatment with CRHR1 antagonists.

Another aspect of the invention concerns a method of identifying a patient with depressive symptoms and/or anxiety symptoms as eligible for a therapy with a CRHR1 antagonist, comprising:

(a) determining in a nucleic acid sample isolated from a patient's sample the status of a biomarker as defined above;

(b) identifying the patient as eligible for a therapy with a CRHR1 antagonist, where the patient's sample is classified as showing the presence of indicator nucleotides as defined above.

In some embodiments of the methods of identifying a patient with depressive symptoms and/or anxiety symptoms as eligible for a therapy with a CRHR1 antagonist, the status of at least 15, at least 20, at least 25 or all of the biomarkers as defined above is determined in a nucleic acid isolated from a patient's sample.

In some embodiments of the above methods of identifying a patient with depressive symptoms and/or anxiety symptoms as eligible for a therapy with a CRHR1 antagonist, the method may further comprise a step of administering a CRHR1 antagonist. The CRHR1 antagonist may be a class I or a class II antagonist.

Most of the non-peptidic CRHR1 antagonists can be described by or adhere to a pharmacophore model that comprises or features a lipophilic top group, a heterocyclic core containing an invariable hydrogen bond acceptor, which is almost always a heterocyclic nitrogen, and a lipophilic, usually aromatic, bottom group.

Class I CRHR1 antagonists as used herein are characterized in that the heterocyclic hydrogen bond acceptor and the bottom group are connected by a two-atom linker as exemplified by CRHR1 antagonists R-121919, NBI-30545, CP-154526, DMP696, pexacerfont (BMS-562086), emicerfont (GW876008), or verucerfont (GSK561679). Class II CRF1R antagonists as used herein are characterized by a two-atom linker between hydrogen bond acceptor and the bottom group as present in CRHR1 antagonist SSR125543A.

In some embodiments of the above methods of identifying a patient with depressive symptoms and/or anxiety symptoms as eligible for a therapy with a CRHR1 antagonist, the CRHR1 antagonist may be selected from the group consisting of CP154,526, Antalarmin, CRA 5626, Emicerfont, DMP-696, DMP-904, DMP-695, SC-241, BMS-561388, Pexacerfont, R121919, NBI30545, PD-171729, Verucerfont, NBI34041, NBI35965, SN003, CRA0450, SSR125543A, CP-316,311, CP-376,395, NBI-27914, ONO-2333Ms, NBI-34101, PF-572778, GSK561579 and GSK586529.

The corresponding structural formulas of some of the above-mentioned CRHR1 antagonists for use in the present invention are set out in Table 2 below:

TABLE 2

| | CRHR1 antagonist | |
|---|---|---|
| Structural formula | (name) | |
| 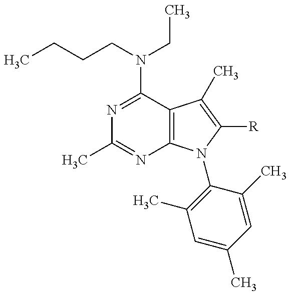 | R = H R = CH₃ | CP154,526 Antalarmin |
| 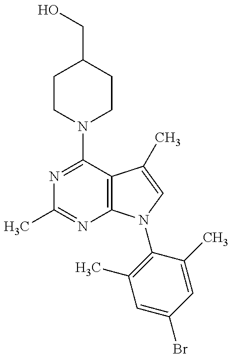 | CRA5626/R317573/ JNJ19567470/ TAI-041 | |
| 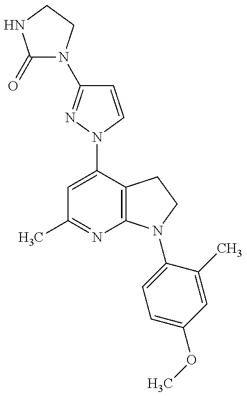 [SMILES: COc1ccc(N2CCc3c(-n4ccc(N5CCNC5=O)n4)cc(C)nc32)c(C)c1] | GW876008/ Emicerfont | |
| 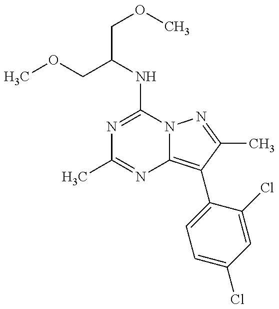 [SMILES: COCC(COC)Nc1nc(C)nc2c(-c3ccc(Cl)cc3Cl)c(C)nn12] | DMP-696 | |
| 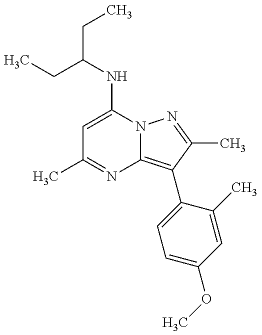 [SMILES: CCC(CC)Nc1cc(C)nc2c(-c3ccc(OC)cc3C)c(C)nn12] | DMP-904 | |
| 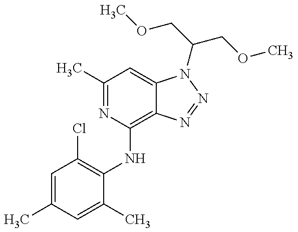 | DMP-695 | |
| 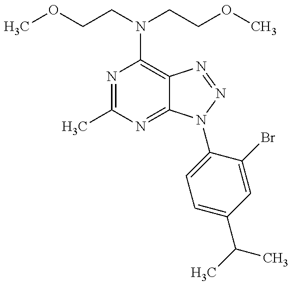 [SMILES: COCCN(CCOC)c1nc(C)nc2c1nnn2-c1ccc(C(C)C)cc1Br] | SC-241/LWH-234 | |
| 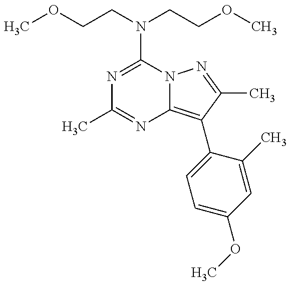 | BMS-561388 | |
| 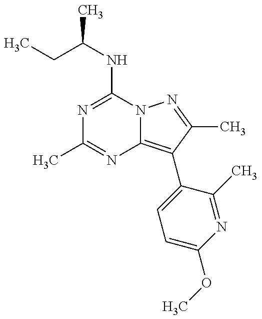 [SMILES: CC[C@@H](C)Nc1nc(C)nc2c(-c3ccc(OC)nc3C)c(C)nn12] | BMS-562086/ Pexacerfont | |
| 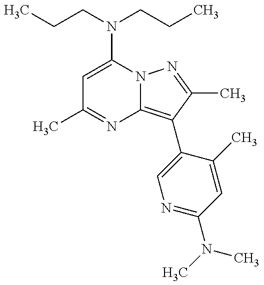 [SMILES: CCCN(CCC)c1cc(C)nc2c(-c3cnc(N(C)C)cc3C)c(C)nn12] | R121919/NBI30775 | |
| 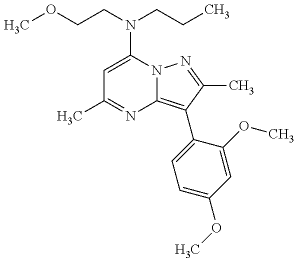 | NBI30545 | |
| 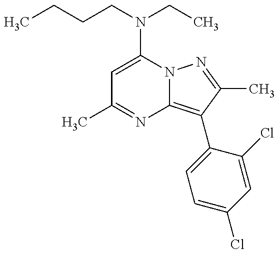 [SMILES: CCCCN(CC)c1cc(C)nc2c(-c3ccc(Cl)cc3Cl)c(C)nn12] | PD-171729 | |
| 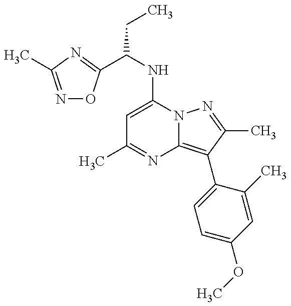 | GSK561679/ NBI-77860/ Verucerfont | |
| 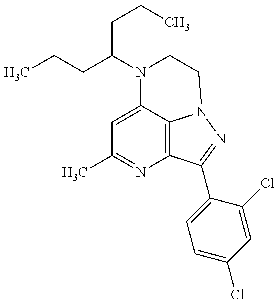 [SMILES: CCCC(CCC)N1CCn2nc(-c3ccc(Cl)cc3Cl)c3nc(C)cc1c32] | SB-723620/ NBI34041 | |
| 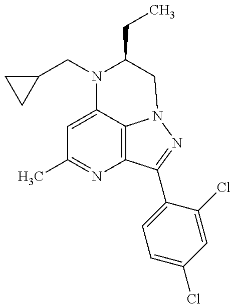 [SMILES: CC[C@H]1Cn2nc(-c3ccc(Cl)cc3Cl)c3nc(C)cc(c32)N1CC1CC1] | NBI35965 | |
| 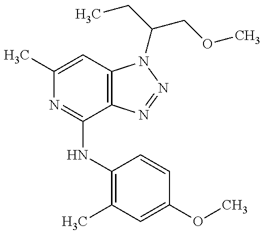 | SN003 | |
| 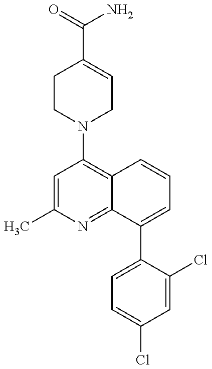 [SMILES: Cc1cc(N2CC=C(C(N)=O)CC2)c2cccc(-c3ccc(Cl)cc3Cl)c2n1] | CRA0450/ R278995 | |
| 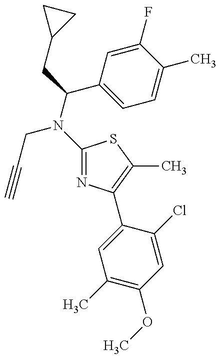 [SMILES: C#CCN(c1nc(-c2cc(C)c(OC)cc2Cl)c(C)s1)[C@@H](CC1CC1)c1ccc(C)c(F)c1] | SSR125543A | |
| 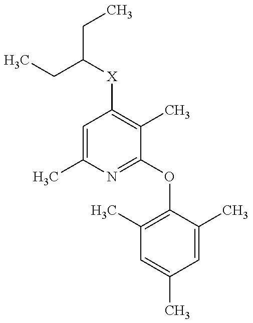 | X = O X = NH | CP-316,311 CP-376,395 |

| | |
|---|---|
| 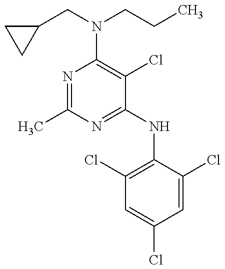 | NBI-27914 |

ONO-2333Ms, NBI 34101, PF-572778, GSK561579 and GSK586529 are described by Zorilla and Koob (Drug Discovery Today, 2010, 371-383) as corticotropin releasing factor receptor antagonists (corticotropin releasing factor is a synonym for CRHR1 antagonists) tested in clinical trials.

Another aspect of the present invention concerns a composition for the analysis of at least one single nucleotide polymorphic indicative for the treatment response to CRHR1 antagonists in patients with depressive symptoms and/or anxiety symptoms, comprising a nucleic acid affinity ligand for a biomarker as defined herein.

The term “nucleic acid affinity ligand” as used herein refers to a nucleic acid molecule being able to bind to a polymorphic site as defined herein. For example, the affinity ligand is able to bind a nucleic acid molecule comprising the sequence of any one of SEQ ID NO: 1 to 30, or fragments thereof, which comprise the polymorphic site as defined herein above, wherein said sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 comprises the respective indicator nucleotide as described herein. In further embodiments of the present invention the nucleic acid affinity ligand may also be able to specifically bind to a nucleic acid molecule, e.g. a DNA molecule comprising, essentially consisting of or consisting of a sequence being at least 90%, 91%, 92%, 93%, 94%, 95%, 96%, 97%, 98% or 99% or 99.5% or 99.6%, 99.7%, 99.8%, or 99.9% identical to the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or fragments thereof, which comprise the polymorphic site as defined herein above, wherein said sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 comprises the respective indicator nucleotide(s) as described herein above, or to any fragments of said sequences.

In further embodiments of the present invention a nucleic acid affinity ligand according to the present invention may also be able to specifically bind to a nucleic acid molecule comprising the sequences of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, with the exception that the sequences do not comprise the indicator nucleotide sequence as defined herein above, i.e. to corresponding wildtype sequences which do not comprise the respective indicator nucleotide as described herein above. In further embodiments of the present invention the nucleic acid affinity ligand may also be able to specifically bind to a nucleic acid molecule comprising, essentially consisting of, or consisting of a sequence being at least 90%, 91%, 92%, 93%, 94%, 95%, 96%, 97%, 98% or 99% or 99.5% or 99.6%, 99.7%, 99.8%, or 99.9% identical to the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 with the exception that the sequences do not comprise the indicator nucleotide sequence as defined herein above, i.e. to corresponding wildtype sequences which do not comprise the respective indicator nucleotide as described herein above, or fragments thereof. In even further embodiments, the present invention relates to nucleic acid affinity ligands binding a nucleic acid molecule comprising a sequence complementary to any one of the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or fragments thereof, which comprises the polymorphic site as defined herein above, which may comprise or may not comprise the indicator nucleotide.

The composition according to the present invention may additionally comprise further ingredients necessary or useful for the detection of protection against drug-resistant epilepsy, such as buffers, dNTPs, a polymerase, ions like bivalent cations or monovalent cations, hybridization solutions, etc.

In yet another preferred embodiment of the present invention the affinity ligand as mentioned herein above may be an oligonucleotide specific for one or more polymorphic sites as defined herein above, or a probe specific for one or more polymorphic sites as defined herein above. The term “oligonucleotide specific for one or more polymorphic sites” as used herein refers to a nucleic acid molecule, preferably a DNA molecule of a length of about 12 to 38 nucleotides, preferably of about 15 to 30 nucleotides. The oligonucleotide may have, for example, a length of 16, 17, 18, 19, 20, 21, 22, 23, 24, 25, 26, 27, 28, 29, or 30 nucleotides. These molecules may preferably be complementary to at least 2, 3, 4, 5, 6, 7, 8, 9, or 10 nucleotides on or around the indicator nucleotide(s) of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, comprising the complementary sequence of said indicator nucleotide(s) as defined herein above in connection with SEQ ID NO: 1 to 30. In further embodiments, the molecules may preferably be complementary to at least 2, 3, 4, 5, 6, 7, 8, 9, or 10 nucleotides on or around the polymorphic site as defined herein above in connection with SEQ ID NO: 1 to 30, however comprising the wildtype sequence.

In other embodiments of the present invention said oligonucleotide as defined herein above may have a sequence complementary to a sequence including the indicator nucleotide(s) of the SNPs of the present invention as defined herein above. In further embodiments the oligonucleotide may also have a complementary sequences towards the counter strand of said sequence including the indicator nucleotide of the SNPs of the present invention as defined herein above.

In further embodiments the present invention also relates to oligonucleotide molecules specifically binding in the vicinity of the polymorphic site as indicated herein above in the context of SEQ ID NO: 1 to 30. These oligonucleotides may be designed in the form of a pair of primers allowing the amplification of stretch of DNA, e.g. of a length of 50 bp, 75 bp, 100 bp, 150 bp, 200 bp, 250 bp, 300 bp, 400 bp, 500 bp, 750 bp, 1000 bp, or more around and including the polymorphic site of the SNPs of the present invention. Suitable sequence information may be derived from the sequence of SEQ ID NO: 1 to 30, the herein above indicated genomic sequence localization, which allows the skilled person to obtain the necessary context DNA sequence from data repositories.

The term “probe specific for one or more polymorphic sites” as used herein refers piece of DNA, which is capable of specifically binding to a polymorphic site according to the present invention. The probe may, for example, be designed such that it only binds to a sequence comprising the indicator nucleotide, or the wildtype sequence, or a complementary strand thereof. In other embodiments the probe may be capable of binding to a polymorphic site according to the present invention, i.e. be able to bind to the wildtype sequence, the indicator nucleotide comprising sequence or any other variant at that position as defined herein above. The specificity of the probe may further be adjusted, for example in hybridization experiments, by the changing the concentration of salts, modifying the temperature of the reaction, adding further suitable compounds to the reaction etc. The probe may also be designed such that it binds outside of the polymorphic site, e.g. within the sequence of SEQ ID NO: 1 to 30.

The probe according to the present invention may, in further embodiments, comprise, essentially consist of, or consist of a nucleic acid molecule being at least 90%, 91%, 92%, 93%, 94%, 95%, 96%, 97%, 98% or 99% or 99.5% or 99.6%, 99.7%, 99.8%, or 99.9% identical to the sequence of SEQ ID NO: 1 to 30, or to fragments thereof, which comprise the polymorphic site as defined herein above, wherein said sequence of SEQ ID NO: 1 to 30 comprises the respective indicator nucleotide as described herein above, or to any fragments of said sequences, or to the corresponding wildtype sequences as defined herein above, or to the complementary sequences of these sequences.

A probe according to the present invention may have any suitable length, e.g. a length of 15, 20, 30, 40, 50, 100, 150, 200, 300, 500, 1000 or more than 1000 nucleotides. The probe may further be suitable modified, e.g. by the addition of labels, e.g. fluorescent labels, dyes, radioactive labels etc.

In further embodiments, the probe may also be functionally adjusted to a detection method.

In yet another aspect the present invention relates to a kit, comprising an oligonucleotide specific for one or more polymorphic sites as defined herein above, or a probe specific for one or more polymorphic sites as defined herein above.

In further embodiments the kit as defined herein above may comprise accessory ingredients such as PCR buffers, ions like bivalent cations or monovalent cations, hybridization solutions etc. The kit may comprise an enzyme for primer elongation, nucleotides and/or lablelling agents. An enzyme for primer elongation may, for example, be a polymerase such as Taq polymerase, Pfu polymerase etc. Nucleotides may preferably be dNTPs, or derivatives thereof. A labeling agent may be, for example, an agent leading to the labeling with a radioactive label, an enzymatic label, a fluorescent label, a chemiluminescent or a bioluminescent label. The term “enzymatic label” relates to labels, which comprise enzymatic activities. A typical, preferred example is the horseradish peroxidase enzyme (HRP). This enzyme complex subsequently may catalyze the conversion of a suitable substrate, e.g. a chemiluminescent substrate into a sensitized reagent which ultimately lead to the emission of light or production of a color reaction. The term “radioactive label” relates to labels emitting radioactive radiation, preferably composed of radioactive isotopes. The term “radioactive isotope” in the context of the label relates to any such factor known to the person skilled in the art. More preferably, the term relates to ³H, ¹⁴C, ³²P, ³³P, ³⁵S or ¹²⁵I. The term “chemiluminescent label” relates to a label, which is capable of emitting light (luminescence) with a limited emission of heat as the result of a chemical reaction. For example, the term relates to luminol, cyalume, oxalyl chloride, TMAE (tetrakis(dimethylamino)ethylene), pyragallol, lucigenin, acridinumester or dioxetane. The term “bioluminescent label” relates to a label, which is capable of emitting light due to a biochemical reaction. Typically, the term refers to the production of light due to the reaction of a luciferin and a luciferase. In such a reaction scheme, the luciferase catalyzes the oxidation of luciferin resulting in light and an inactive oxyluciferin. The term “fluorescent label” relates to chemically reactive derivatives of a fluorophores. Typically common reactive groups include amine reactive isothiocyanate derivatives such as FITC and TRITC (derivatives of fluorescein and rhodamine), amine reactive succinimidyl esters such as NHS-fluorescein, and sulfhydryl reactive maleimide activated fluors such as fluorescein-5-maleimide. Reaction of any of these reactive dyes with another molecule results in a stable covalent bond formed between a fluorophore and a labelled molecule. Following a fluorescent labeling reaction, it is often necessary to remove any nonreacted fluorophore from the labeled target molecule.

In further embodiments the kit may also comprise accessory ingredients like secondary affinity ligands, e.g. secondary antibodies, detection dyes, or other suitable compound or liquids necessary for the performance of a nucleic acid detection. Such ingredients as well as further details would be known to the person skilled in the art and may vary depending on the detection method carried out. Additionally, the kit may comprise an instruction leaflet and/or may provide information as to the relevance of the obtained results.

In yet another aspect the present invention relates to a microarray, comprising at least one probe selective for an indicator nucleotide or the corresponding wildtype nucleotide as defined herein above. In a standard setup such a microarray comprises immobilized probes to detect a nucleic acid comprising a polymorphic site as defined herein above. The probes on the array may, for example, be complementary to one or more parts of the sequence of SEQ ID NO: 1 to 30 and/or to corresponding wildtype sequences. Typically, cDNAs, PCR products, and oligonucleotides may be used as probes. The microarray may comprise probes of one or more SEQ ID NO: 1 to 30, or corresponding wildtype sequences, or all of these biomarkers or any combination of said markers. Furthermore, any type of fragment or sub-portion of any of the markers sequences may be combined with any further fragment or sub-portion of any of said sequences SEQ ID NO: 1 to 30, or corresponding wildtype sequences.

There is virtually no limitation on the number of probes which are spotted on a DNA array. Also, a marker can be represented by two or more probes, the probes hybridizing to different parts of a gene. Probes are designed for each selected marker gene. Such a probe is typically an oligonucleotide comprising 5-50 nucleotide residues. Longer DNAs can be synthesized by PCR or chemically. Methods for synthesizing such oligonucleotides and applying them on a substrate are well known in the field of micro-arrays.

The methods described above are not restricted to methods related to a treatment response to CRHR1 antagonists in patients with depressive symptoms and/or anxiety symptoms. The treatment response to any other compound, drug or biomolecule that is capable for treating depressive symptoms and/or anxiety symptoms in patients who have CRH overactivity may be also be predicted by methods described herein. In particular, the disclosure can be understood to mean that the term “CRHR1 antagonists” can be replaced by any other compound that interferes with the CRHR1 pathway and leads to a remission of depressive symptoms and/or anxiety symptoms patients with CRH overactivity.

The invention is further described in the following examples which are solely for the purpose of illustrating specific embodiments of the invention, and are also not to be construed as limiting the scope of the invention in any way.

### Example 1

### Methods

Patients:

Patients with unipolar or bipolar depression admitted as in-patients to the Max Planck Institute of Psychiatry (MPI), Munich, Germany, for treatment of a depressive episode were included in the study. Patients were diagnosed by psychiatrists according to the Diagnostic and Statistical Manual of Mental Disorders (DSM) IV criteria. Patients with bipolar disorder or depressive disorder due to a general medical or neurological condition were excluded, as were patients with a lifetime diagnosis of drug abuse and depressive symptoms secondary to alcohol or substance abuse or dependency. Ethnicity was recorded using a self-report sheet for nationality, first language and ethnicity of the patient and of all four grandparents. All patients were Caucasian and part of the Munich-Antidepressant-Response-Signature (MARS) project (Hennings et al., J Psychiatr Res., 2009) (www.mars-depression.de). They were treated with antidepressant medications according to doctor's choice. Severity of depressive symptoms was assessed at admission and at the time of the dex/CRH test by trained raters using the 17-item Hamilton Depression Rating Scale (HAM-D, Hamilton, J Neurol Neurosurg Psychiatry, 1960). 192 patients fulfilling the criteria for at least a moderate to severe depressive episode (HAM-D≧18) at both time points and who had been administered a dex/CRH test within 10 days of in-patients admission and had genome-wide SNP data were included in this analysis. The study was approved by the Ethics Committee of the Ludwig Maximilians University in Munich, Germany, and written informed consent was obtained from all subjects.

Dex/CRH Test:

The dex/CRH test was administered as described in detail in Heuser et al. (J Psychiatr Res., 1994). In brief, subjects were pre-treated with 1.5 mg of dexamethasone per os at 11 pm. The following day, at 3 pm, 3.30 pm, 3.45 pm, 4 pm and 4.15 pm blood was drawn. An intravenous bolus of 100 μg of human CRH (Ferring, Kiel, Germany) was given at 3.02 pm. Plasma ACTH concentrations were assessed by an immunometric assay without extraction (Nichols Institute, San Juan Capistrano, Calif.; USA). The neuroendocrine response to the dex/CRH test was analyzed using the total area under the curve (AUC) of the ACTH response.

SNP Genotyping:

After enrollment in the study 40 ml of EDTA blood was drawn from each patient. DNA was extracted from fresh blood using the Puregene® whole blood DNA-extraction kit (Gentra Systems Inc; MN). Genotyping was performed on Illumina Human 610k quad genotyping arrays (Illumina Inc., San Diego, USA) according to the manufacturer's standard protocols. The average call rate exceeded 99%, with samples below 98% being either retyped or excluded from the study. The reproducibility for samples genotyped twice was 99.99% or better.

Data Analysis:

To identify genetic predictors for the ACTH response to the dex/CRH test in patients with moderate to severe depression, the 192 patients were randomly split into a trainings (N=96) and test set (N=96). The demographic and clinical descriptors of these two samples are given in table 2.

TABLE 2
Demographic and clinical description of the 192 patients in the
trainings and test set.

| | training set | | test set | | p-value |
|---|---|---|---|---|---|
| N | 96 | | 96 | | |
| % female | 52.1 | | 57.2 | | n.s. |
| % bipolar (1 or 2) | 12.5 | | 12.5 | | n.s. |
| % with psychotic | 15.2 | | 11.4 | | n.s. |
| symptoms | | | | | |
| age (mean years (SD)) | 47.2 | (14.2) | 51.3 | (13.2) | 0.038 |
| BMI at admission | 24.9 | (4.1) | 24.9 | (4.3) | n.s. |
| (mean SD) | | | | | |
| age-on (mean years (SD)) | 34.72 | (14.5) | 36.6 | (15.9) | n.s. |
| number of previous | 3.2 | (4.0) | 3.3 | (4.7) | n.s. |
| episodes (mean N (SD)) | | | | | |
| depression severity at | 26.4 | (4.4) | 27.7 | (4.8) | 0.058 |
| admission (mean HAMD- | | | | | |
| 17 score (SD)) | | | | | |
| depression severity at | 26.9 | (5.0) | 28.5 | (5.0) | 0.029 |
| dex-crh test (mean | | | | | |
| HAMD-17 score (SD)) | | | | | |
| Cortisol AUC (mean (SD)) | 3611.4 | (3414) | 3688.6 | (3491) | n.s. |
| ACTH AUC (mean (SD)) | 1246 | (910) | 1482.8 | (1319) | n.s. |
| days after admission when | 6.1 | (2.2) | 5.9 | (2.2) | n.s. |
| dex-CRH test was | | | | | |
| performed | | | | | |

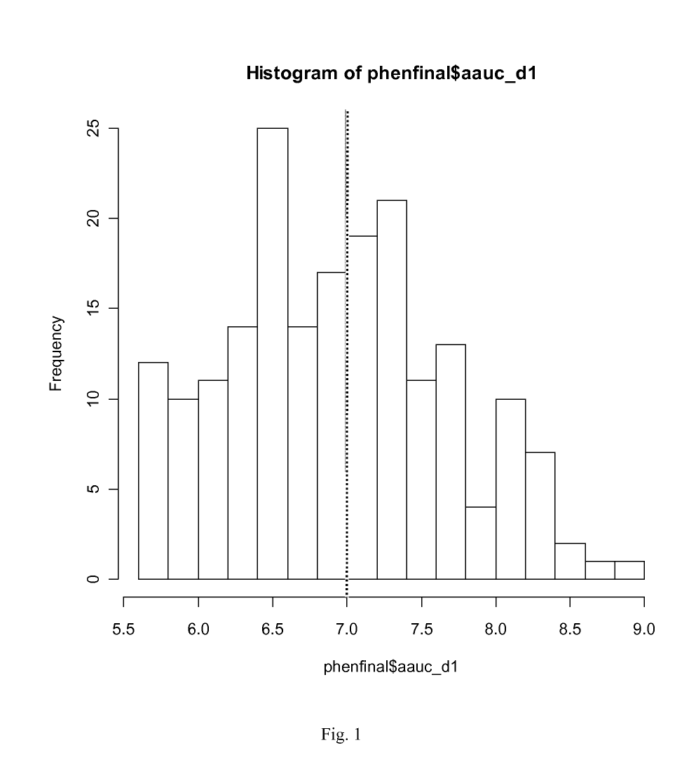

After natural log transformation of the AUC of the ACTH response in the dex/CRH test, patients were dichotomized into high vs. low responders. For the ACTH AUC the cut-off was ln ACTH AUC>7.0. This placed 46.4% (89 out of a total of 192 individuals) in the high responder class. See the corresponding histogram in FIG. 1.

Using an additive genetic model and a logistic regression with sex and age as covariates in PLINK (http://pngu.mgh.harvard.edu/purcell/plink/), the association of all SNPs with a MAF>0.05 were tested on the 610k arrays with the dichotomized response for cortisol and ACTH in the dex/CRH test in the training set. The top 30 associated SNPs were then used to predict either ACTH or cortisol response status in the test set using a “Probabilistic Neural Network and General Regression Neural Network” approach (implementation DTREG 10.3.3 www.dtreg.com). All values were derived from a 20 fold cross validation.

Results:

The top 30 association with ACTH response status are given in table 3. Association p-values ranged from 5.0 e-5 to 2.3 e-4 for the ACTH response.

The genotypes for the 30 SNPs each were then used to predict ACTH response status in the second, independent test cohort (subgroup of the test set).

TABLE 3
List of 30 SNPs used to predict ACTH AUC in the second test cohort.

| | | | P-value | | |
|---|---|---|---|---|---|
| SNP | Chromosome | Coordinate_HG18 | training | GeneVariant | GeneName |
| rs6437726 | chr3 | 108321446 | 5.037e−005 | INTERGENIC | N/A |
| rs1986684 | chr11 | 110629697 | 5.287e−005 | UPSTREAM | N/A |
| rs7380830 | chr5 | 66022641 | 5.459e−005 | INTRONIC | ENSG00000196567 |
| rs3903768 | chr13 | 49294756 | 6.811e−005 | INTERGENIC | N/A |
| rs7325978 | chr13 | 71960761 | 8.206e−005 | INTERGENIC | N/A |
| rs13585 | chr22 | 45131843 | 0.0001016 | REGULATORY_REGION, | TRMU |
| | | | | 3PRIME_UTR | |
| rs9368373 | chr6 | 22024631 | 0.0001063 | INTERGENIC | N/A |
| rs10935354 | chr3 | 141026326 | 0.0001073 | INTERGENIC | N/A |
| rs8095703 | chr18 | 11041081 | 0.0001183 | INTERGENIC | N/A |
| rs10206851 | chr2 | 5381477 | 0.0001267 | INTERGENIC | N/A |
| rs9542977 | chr13 | 71958236 | 0.0001499 | INTERGENIC | N/A |
| rs4942879 | chr13 | 49314940 | 0.0001537 | INTERGENIC | N/A |
| rs9542954 | chr13 | 71918308 | 0.0001643 | INTERGENIC | N/A |
| rs1593478 | chr18 | 2292023 | 0.0001663 | INTERGENIC | N/A |
| rs9542951 | chr13 | 71913959 | 0.0001664 | INTERGENIC | N/A |
| rs2188534 | chr7 | 111837550 | 0.0001823 | INTERGENIC | N/A |
| rs12524124 | chr6 | 22051921 | 0.000185 | INTERGENIC | N/A |
| rs4352629 | chr5 | 87792577 | 0.0001985 | INTERGENIC | N/A |
| rs7448716 | chr5 | 87788451 | 0.0001985 | INTERGENIC | N/A |
| rs11873533 | chr18 | 3732713 | 0.0002107 | INTRONIC | DLGAP1 |
| rs10062658 | chr5 | 102881920 | 0.0002216 | INTERGENIC | N/A |
| rs12547917 | chr8 | 142306580 | 0.0002282 | INTRONIC | SLC45A4 |
| rs1038268 | chr9 | 31967623 | 0.000229 | INTERGENIC | N/A |
| rs2375811 | chr9 | 31979998 | 0.000229 | INTERGENIC | N/A |
| rs1352671 | chr9 | 32033985 | 0.000229 | INTERGENIC | N/A |
| rs364331 | chr9 | 32039631 | 0.000229 | INTERGENIC | N/A |
| rs1924949 | chr13 | 71975473 | 0.0002323 | INTERGENIC | N/A |
| rs11025990 | chr11 | 21218608 | 0.0002323 | INTRONIC | NELL1 |
| rs3758562 | chr10 | 72032086 | 0.0002369 | INTRONIC | PRF1 |
| rs10156056 | chr7 | 22720613 | 0.0002394 | INTERGENIC | N/A |

The results of the prediction in the test set are summarized below:

For the prediction of the dichotomized ACTH response status in the dex/CRH the following prediction values were achieved:

ACTH:
Accuracy=85.33%
Sensitivity=87.18%
Specificity=83.33%
Geometric mean of sensitivity and specificity=85.23%
Positive Predictive Value (PPV)=85.00%
Negative Predictive Value (NPV)=85.71%
Geometric mean of PPV and NPV=85.36%
Precision=85.00%
Recall=87.18%
F-Measure=0.8608
Area under ROC curve (AUC)=0.851852

Using genome-wide SNP association data for the ACTH response in the dex/CRH test, a subset of 30 SNPs could be identified that allowed an accurate, sensitive and specific prediction of these phenotypes in independent sets of patients. Increased ACTH secretion in this test has been linked to an increase in central CRH/CRHR1 function. Although environmental, pharmacological and disease state-dependent factors have previously been described to influence the endocrine response to this test in depressed patients (Ising et al., Neuropsychopharmacol Biol Psychiatry, 2005; Heim et al. Biol Psychiatry, 2008; Kunzel et al. Neuropsychopharmacology, 2003), it has now been shown that genetic polymorphisms alone are strong predictors.

Patients with depression or anxiety disorders, classified into the high ACTH response group according to the genotypes of the presented 30 SNPs described in this example will be more likely to respond to CRHR1 antagonist treatment. This allows an enrichment of such patients for CRHR1 antagonist treatment studies who will most likely respond to this specific treatment.

### Example 2

Sleep disturbances, such as decreased slow-wave sleep, increased sleep fragmentation and rapid-eye-movement sleep (REMS) disinhibition, are cardinal symptoms of major depression in humans. This study aims to identify those patients where a central CRH hyperdrive plays a causal role and which would therefore respond favourably to a CRHR1 antagonist. To test the relationship between a central CRH-overexpression and REM-disinhibition in particular, transgenic mouse models where CRH is overexpressed as a result of genetic engineering were employed.

Many animal models of depression share increases in REM-sleeps (REMS) as a common feature. Therefore, increased REMS in animals should reflect REMS-disinhibitions in humans. Mice with CNS-specific CRH-overexpression strikingly share the characteristic increases in REMS. As such, an increase in REMS indicates a central hypersecretion of CRH and may serve as a biomarker to identify those patients who would benefit from treatment with a CRHR1 antagonist.

Experiments were conducted with two different mouse lines of excessive central CRH secretion and their respective control littermates (CL). Mice of the CRH-COE^{CNS} line are characterised by CRH-overexpression within the whole CNS, whereas mice of the Cor26 CRH line display a CRH-overexpression specific to CRH-ergic neurons of the CNS. Three different CRHR1 antagonists were tested. While DMP-696 (bicyclic) and CP-316,311 (monocyclic) are class I CRH-R1 antagonists, SSR125543A (long off-rate, typical slow-tight binding inhibitor) belongs to class II CRH-R1 antagonists. DMP-696 and SSR125543A were applied to CRH-COE^{CNS} mice (n_{DMP696}=6/6 COE/CL; n_{SSR125543}=6/5 COE/CL), while CP-316,311 was tested in Cor26 CRH mice (n_{CP316.311}=5/3 Cor26/CL). In all cases, animals were left to recover from EEG/EMG-electrode implantation for two weeks, after which two days of baseline recording were initiated. Treatment with CRH-R1 antagonist or respective vehicle control commenced thereafter for five consecutive days. Antagonists were applied through the drinking water at a daily dose of 50 mg/kg body weight. EEG and EMG recordings were manually scored as wake, non-REMS (NREMS), and REMS in four second epochs by an experienced evaluator.

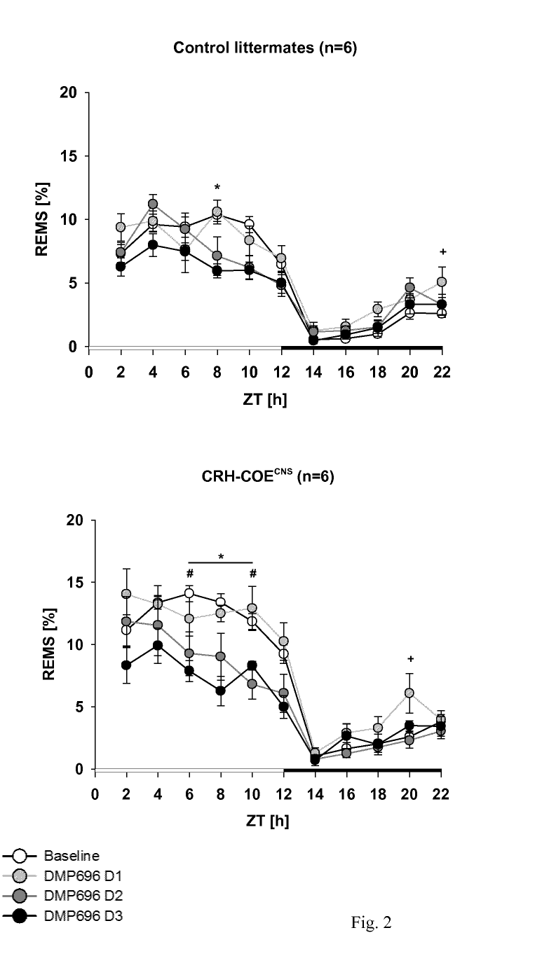

As previously shown, CRH-COE^{CNS} mice display significantly higher REMS activity under baseline condition as compared to controls. Chronic DMP-696 (50 mg/kg/d DMP-696) treatment entails only a mild suppression of REMS in CL mice. However, DMP-696-treated CRH-COE^{CNS} mice show a significant decrease in REMS activity beginning with treatment day two (P<0.05). The strongest suppression of REMS activity in CRH-COE^{CNS} animals could be observed on treatment day three (FIG. 2).

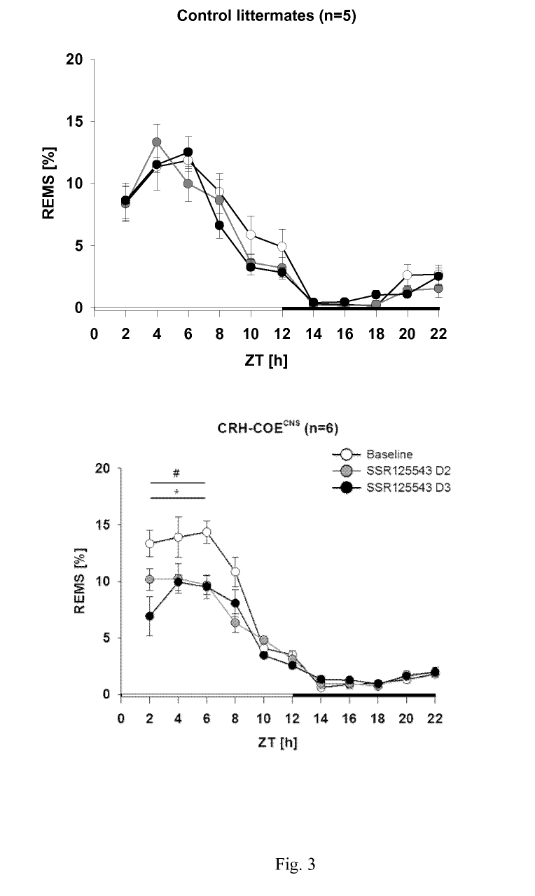

Comparable to DMP-696 treatment, oral application of SSR125543A (50 mg/kg/d) affected REMS levels in CRH-overexpressing mice. No effects of SSR125543 on REMS activity in control animals could be detected. In contrast, a significant suppression of REMS could be observed beginning with day two in CRH-overexpressing animals (P≦0.035). Similar to DMP-696 treatment, REMS suppression in CRH-COE^{CNS} mice never exceeded baseline REMS-levels of CL (FIG. 3).

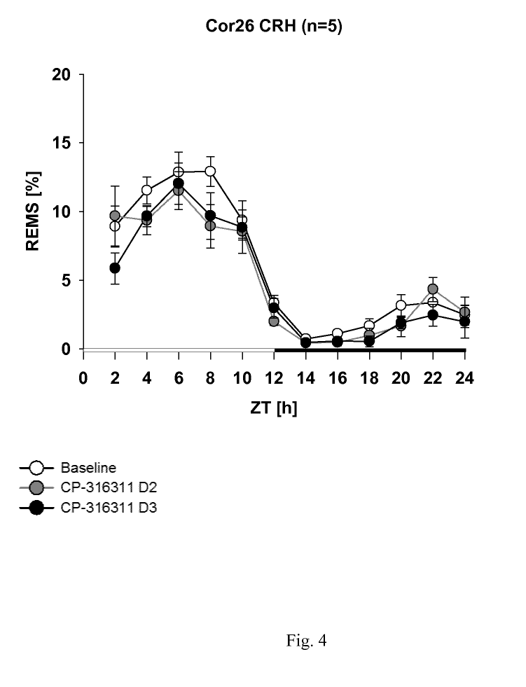

Application of CP-316,311 (Pfizer) in the Cor26 CRH mouse line showed no significant effect on REMS levels in CL animals. Similarly, in CRH-overexpressing Cor26 CRH mice suppression in REMS apparently seemed weak. However, comparison of the area under the curve (AUC) within the light period of baseline and treatment day three showed a significant decrease (P=0.006) of REMS levels after CP-316,311 application (FIG. 4).

CRH is one of the major drivers of the stress response in the brain. Hyperactivity of the CRH system seems to be responsible for cognitive impairments, emotional responses, and behavioural changes which are typical for depression. One of those behavioural changes are sleep disturbances exemplified by REMS disinhibition. The link between CRH-overexpression and REMS level increases is evidenced by the mouse lines used in these experiments. Since CRH-overexpression in the Cor26 CRH mouse line is limited to CRH-ergic neurons, the net increase of CRH is lower when compared to the whole brain overexpression in CRH-COE^{CNS} mice. As a result, the phenotype of increased REMS levels was less profound in Cor26 CRH mice as compared to CRH-COE^{CNS} animals.

The main finding of this study is that the normalization of CRH-elicited sleep-EEG disturbances is striking when (1) different chemical classes of CRHR1 antagonists are used and (2) different animal models for CRH-induced sleep-EEG changes that are typical for human depression are employed. REMS disinhibition is indicative of a central CRH dysfunction (i.e. hyperactivity) and as such may serve as a biomarker for the identification of depressed patients where depression is caused by central CRH-hyperdrive. Normalization of the sleep pattern by application of different CRHR1 antagonists could be shown in all of our experiments, employing different classes of CRHR1 receptors and different animal models overexpressing centrally CRH.

1. A method for providing an algorithm for predicting a treatment response to CRHR1 antagonists in patients with depressive symptoms and/or anxiety symptoms, wherein the method comprises the following steps:
(a) performing a single nucleotide polymorphism (SNP) genotyping analysis in a group of patients with depressive symptoms and/or anxiety symptoms;
(b) determining a value indicative for CRH activity in each patient of the group, wherein a value indicative for CRH overactivity is indicative for a patient responding to a treatment with a CRHR1 antagonist;
(c) identifying at least one SNP associated with a value indicative for CRH overactivity as determined in step (b); and
(d) determining the algorithm by machine-learning from the association of the at least one SNP identified in step (c) with the value indicative for CRH overactivity.

2. The method according to claim 1, wherein the SNP genotyping analysis is performed in a group of at least 10 patients.

3. The method according to claim 1, wherein the SNP genotyping analysis comprises the use of SNP-specific primers, SNP-specific probes, a primer extension reaction, the use of SNP microarrays and/or the use of sequencing methods.

4. The method according to claim 1, wherein the value indicative for CRH activity in each patient is determined by measuring ACTH response to a combined dexamethasone suppression/CRH stimulation test in each patient.

5. (canceled)

6. (canceled)

7. The method according to claim 1, wherein in step (d) a number N of SNPs identified in step (c) is associated with a value indicative for CRH overactivity, wherein N is sufficient to provide an algorithm having an accuracy of prediction of at least 80% and/or a sensitivity of prediction of at least 70% and/or a specificity of prediction of at least 70% and/or a positive predictive value of prediction of at least 70% and/or a negative predictive value of prediction of at least 70%.

8. The method according to claim 7, wherein N is at least 20.

9. The method according to claim 1, wherein step (c) further comprises identifying at least one SNP associated with a value indicative for normal CRH activity as determined in step (b), and step (d) further comprises machine-learning from the association of the at least one SNP associated with the value indicative for normal CRH activity identified in step (c) with the value indicative for normal CRH activity.

10. The method according to claim 1, wherein the algorithm determined in step (d) associates at least one SNP selected from the group consisting of SNPs described in table 1 and an SNP in strong linkage disequilibrium with an SNP described in table 1 with a value indicative for CRH overactivity or with a value indicative for normal CRH activity.

11. The method according to claim 1, wherein the algorithm determined in step (d) associates the SNPs described in table 1 with a value indicative for CRH overactivity and with a value indicative for normal CRH activity, respectively.

12. A method for predicting a treatment response to CRHR1 antagonists in patients with depressive symptoms and/or anxiety symptoms, wherein the method comprises the following steps:
(a) determining in a nucleic acid sample obtained from a patient the presence or absence of at least one single nucleotide polymorphism (SNP) associated with a value indicative for CRH overactivity;
(b) predicting the treatment response to CRHR1 antagonists by linking an algorithm provided by the method of claim 1 with the presence or absence of the at least one SNP determined in step (a).

13. The method according to claim 12, wherein step (a) comprises determining at least one of the SNPs, which were associated with a value indicative for CRH overactivity when determining the algorithm by machine-learning from this association.

14. The method according to claim 12, wherein step (a) is preceded by a step of obtaining a nucleic acid sample from a patient.

15. The method according to claim 12, wherein in step (a) the presence or absence of a number N of SNPs is determined.

16. The method according to claim 15, wherein N is at least 20.

17. The method according to claim 12, wherein determining in step (a) further comprises determining at least one SNP associated with a value indicative for normal CRH activity.

18. The method according to claim 12, wherein the at least one SNP, determined in step (a) is selected from the group consisting of SNPs described in table 1 and an SNP in strong linkage disequilibrium with an SNP described in table 1.

19. The method according to claim 12, wherein determining in step (a) comprises determining the SNPs described in table 1.

20. The method according to claim 12, wherein the at least one SNP associated with a value indicative for CRH overactivity or with a value indicative for normal CRH activity is determined in step (a) by using SNP-specific primers, SNP-specific probes, a primer extension reaction, SNP microarrays and/or sequencing methods.

21. A group of biomarkers, comprising:
SNP rs6437726,
SNP rs1986684,
SNP rs7380830,
SNP rs3903768,
SNP rs7325978,
SNP rs13585,
SNP rs9368373,
SNP rs10935354,
SNP rs8095703,
SNP rs10206851,
SNP rs9542977,
SNP rs4942879,
SNP rs9542954,
SNP rs1593478,
SNP rs9542951,
SNP rs2188534,
SNP rs12524124,
SNP rs4352629,
SNP rs7448716,
SNP rs11873533,
SNP rs10062658,
SNP rs12547917,
SNP rs1038268,
SNP rs2375811,
SNP rs1352671,
SNP rs364331,
SNP rs1924949,
SNP rs11025990,
SNP rs3758562, and
SNP rs10156056.

22. (canceled)

23. (canceled)

24. (canceled)

25. A method for detecting CRH overactivity in a patient with depressive symptoms and/or anxiety symptoms, comprising determining the status of a biomarker as defined in claim 21 in a nucleic acid sample isolated from a patient's sample, wherein the presence of indicator nucleotides is indicative for CRH overactivity.

26. A method for monitoring depression and/or anxiety therapy of a patient with a CRHR1 antagonist comprising the step of determining the status of a biomarker as defined in claim 21 before and during the therapy.

27. A method of identifying a patient with depressive symptoms and/or anxiety symptoms as eligible for a therapy with a CRHR1 antagonist, comprising:
(a) determining in a nucleic acid sample isolated from a patient's sample the status of a biomarker selected from the group consisting of SNP rs6437726, SNP rs1986684, SNP rs7380830, SNP rs3903768, SNP rs7325978, SNP rs13585, SNP rs9368373, SNP rs10935354, SNP rs8095703, SNP rs10206851, SNP rs9542977, SNP rs4942879, SNP rs9542954, SNP rs1593478, SNP rs9542951, SNP rs2188534, SNP rs12524124, SNP rs4352629, SNP rs7448716, SNP rs11873533, SNP rs10062658, SNP rs12547917, SNP rs1038268, SNP rs2375811, SNP rs1352671, SNP rs364331, SNP rs1924949, SNP rs11025990, SNP rs3758562, and SNP rs10156056; and
(b) identifying the patient as eligible for a therapy with a CRHR1 antagonist, where the algorithm provided by the method of claim 1 predicts that the patient responds to the treatment with CRHR1 antagonists.

28. (canceled)

29. The method of claim 27, further comprising a step of administering a CRHR1 antagonist.

30. The method of claim 27, wherein the CRHR1 antagonist is selected from the group consisting of CP154,526, Antalarmin, CRA 5626, Emicerfont, DMP-696, DMP-904, DMP-695, SC-241, BMS-561388, Pexacerfont, R121919, NBI30545, PD-171729, Verucerfont, NBI34041, NBI35965, SN003, CRA0450, SSR125543A, CP-316,311, CP-376,395, NBI-27914, ONO-2333Ms, NBI-34101, PF-572778, GSK561579 and GSK586529.

31. (canceled)

32. (canceled)

33. (canceled)

34. A composition for the analysis of at least one single nucleotide polymorphism (SNP) indicative for the treatment response to CRHR1 antagonists in patients with depressive symptoms and/or anxiety symptoms, comprising a nucleic acid affinity ligand for a biomarker as defined in claim 21.

35. A kit, diagnostic composition or device for the analysis of at least one single nucleotide polymorphism (SNP) indicative for the treatment response to CRHR1 antagonists in patients with depressive symptoms and/or anxiety symptoms, comprising at least one primer and/or probe selective for determining the presence or absence of at least one SNP associated with a value indicative for CRH overactivity.

36. (canceled)

37. (canceled)

38. (canceled)

39. (canceled)

40. (canceled)

41. (canceled)


SEQUENCE LISTING (30 sequences)

SEQ ID NO: 1 - DNA, 401 bp, Homo sapiens
caagaaagagagtaataaaaataaccacaatgagggctctcattaatactggatcttatggaaaccaattgttcagtccctcaacaaaagaccagatgggcaggaagctaaatatacaccatgcactaaacattatgagtatcatagtttacaagtcaaagggggctctattgaagatagttctattttccctctatattatctgctagacaatacctgataacattatccaagtaaatgacaacttgataaatagtaatttccaatggtgaacagaggtgacatttcctcattacaaaaatattttctttggcagatgagattaactgaataagaaatccactgacactgaaatcacagagccaaattccctatcacagcacttatcacattgcgttagg

SEQ ID NO: 2 - DNA, 401 bp, Homo sapiens
ttccttgtagcggggagagagactcagggaaggcagggtgatacctgagttggggcttaaagcaaggtagggtgtgtgtggtgatggcaaaataggtaggaagacagcacgggcaaagtcctggaggcaaggacagaaagaggaagtggcaggaagtgaggctggggaaatgagtaggggtcaatcatgatgtttctggtatagggaagagtttggaatgcatcctctaggccatacgccattgggggcttttaagaaagacagtgatgttggtttgatttgcattttatatagacttttctggcagctgagaggaaggtggttttgagaatcacaaagctgcgggaagatcagtcaggagggttctagaataatccaggcaagagctgatggggactgag

SEQ ID NO: 3 - DNA, 401 bp, Homo sapiens
acaggggtggctactctttctccagaaataggtgtcctgtggggcattttgaagtagaatgttgatagttgctttcaattttagactggtaaataagaattgggcatttgaatttcaatatactcactgtgtaactgttattgagtatgctttaagtgacctataatactgcttcatttaactttattgtcctaataactctcttagagtgacaataacttaggttagccacttgcctagggttctgaaaccaagtaaatggtggagctggaattgctgttcttgtcagtcattagactagatcggttttcttcttcctacaaattttatatactaaaaaattttgaaaaaagacatttttctttgggaaaaatagggaatgtcagatccctttggagatg

SEQ ID NO: 4 - DNA, 401 bp, Homo sapiens
ctcgcagcaaccaagcctgcccaagccggggaaacctggggagcaaaccttcacctgcactgtacatcagagaccagttggccctattttggctcctgtggacaggtaagtatcccttttgactcatcccccaaatatcaggtgagccaggaaaataaggcctttggcttagacagtcaattcaaagtctgccatagcatacctaattacatccctattgccccttttctaggtcgtttctcctctaacacgattttatttttctgtcagccattttattttatttctcaccttgaaatatatgttttctttgcagtttttgctttggcttcctgctaactctatttgggcaattgtttaaggctgaacacttggttatgagaggtaccctgttgtgttga

SEQ ID NO: 5 - DNA, 401 bp, Homo sapiens
tcatcaagtctcctttttctctaggaaaaataacattgtcaaggttattaacagtcaataagctgtcataggctcagcatggatggggatattgggtttccttgtgcttatatgaaagatgggaaaatccgaagttcttttcaccctgatatggaaaatacccaacatgaggagaagcagcagctatatgattctgagcacagaatgggagtaagaatagggtcatgctgtactgattatctgctaataaaatgcaaaagtgttaggtaatttcatcaatatccagttaatactaatatagttaatatttcatgactgggtaatattttataatgataaatatttttatagatcttagctctttttattctcatatcaactgtatgaaatcagtgattggt

SEQ ID NO: 6 - DNA, 385 bp, Homo sapiens
ctggggacctcagggagaggtacgcaggttgccatggctgcgtctgcagtccacctgcctttccacgccagggagtcagtgatgtggagccccctgggccccagtggaagcagcgatcagactatgtgtccttgaaataatgtttattccacgctgtcccgacagccccctctgcaggtcccctcggtgtactctgaggtgggaaaccctccctgggggcggtgaaggggaactcgggccaccccaccagccagcagatgctccagcagccagagccccagcctggagctgaggctcttcctggggctcgccgggcccctgcaggcttttcggaccctcagccagcccggcttcctctgctttgggcagcagcaagctggccctt

SEQ ID NO: 7 - DNA, 401 bp, Homo sapiens
ttcctgtgcctcagctcctctgtagaatggtgctggcaatacagtttgcctcattgggctcttgtaagctttaaataggttattatacataaagagctaatagtgatgcctgtagccgttgtctaagtgctagctctgatgatggtgacaaagaagtaatagcaatcagtggtttagattaaaccattttaggcataaaccgttctgctagaatccaaggggagattttttcccatcaaggagacatagcttgttgggaagataagacatacccaattgcagaagtaattaattaattcttttttttttttttttttttttttttgcgatggagtttcgctcttgttgcccaggctggagtgcaatagcatgatctcggctcaccacaacctctgcctcct

SEQ ID NO: 8 - DNA, 401 bp, Homo sapiens
ataggccctatacagctctcaatttctttaatcaatcttcctagcagcccgtgagaaatattactgtcttcagcttcctaaaggagaaaacagaggcctggagggattaaaagacttttctaagattttagagggcatgttagggttcaggcccagggctgtctaacccaaggcctaattccttctattacatccatcatacatgagtgagcactgggcatgaggatacgtcagtgaaaggggccctgtaacatggaccttacattttggctgggggagacaggcaatgaatacataggaccatgttgggaagtgctaagtactctgatgataacacagcagggtgaggtgacagaggtctagggagagtggtgttcagcaaaaacttctctggggagaga

SEQ ID NO: 9 - DNA, 401 bp, Homo sapiens
aaaatttaccaggtttaaaaaaaaaaaaactcaaatgatatttcagaaacctacccctttcaaaacaaggaggagaaaaatcttctccacaaaagcacatattgaaaaaatattttgggggcaaggcctgaaagggttggcagtgtgcagttctgttattattcccgtggccattttatgggcctcagcaaaacactgggatcattatctgtcttctggttactccaggagagctagccatcacaacccaatggaagagacttcagagaaacccacacaggcaccagaagtccttccctttcatctgccactgtggggttttgtcctcatctattacaatgttgtccaatctcagactgcattcagaacaaaggctctcagactgaggatgagttcttgga

SEQ ID NO: 10 - DNA, 401 bp, Homo sapiens
ccaaataattgttattgttgttttaacatggcaatcacgttatttgccatatgtgaaaaagaatatttaaaatgctttttaaaactatgtatgtaaaagaatgtttaaattgttttaaaaatatgttatatctaccttggcaccatccttgctgttgagaaatgacttttacctgcttacttagaaggaaatgtcagaagcagaagtacatttgaatacgattatttgaaagcttcatccatttttcaaagaatgtatacagtaacactaaatagaaagcatagtttatcaactcttcactaagaacagtctagcaagtatatcagagtggctgtggttccagttggactaacctaatcatttatgaaaaggtgataataagcttggaccaagagcaccca

SEQ ID NO: 11 - DNA, 401 bp, Homo sapiens
cagatgttatgtgaaactctggagaaatagtagcaagcaagactcacatgcctcctgccctcacagagctccatgatctggtgaaagtgccagatatttaaacccatggatgtgtgcacacaaaataacaattctctcaagcgttgtgaagaaaagtcacagagcactacaaaagcatgtaagagtgaggcaaaacctatcgtgttaggacagggaaggcttctgtgagctaacctgaaggatgagtaggggtgagccagatgaaaaggcgagagaaaaacattctgagcagagactgccactgagtgcatcccagttttcccaacatcttaacactgtataatgactacactggattttcttcatcctggatccatggttagacatgttaatatgccttc

SEQ ID NO: 12 - DNA, 401 bp, Homo sapiens
cccagtctgtggtatttttttatagcagcacaaacagactaacacaagaggtggataggatttgcgagcatggaccttggaggtttgtggcctcaatttaaagtgagtacattcacccagctggtgtttttctcttgctgcttgggcacagagatggagtaaatgggtctaatcaaggataaagggagagccaaagagatagtaatatttgaaaggaagtgtttttaatgatgtgccatgtaatctgagctgggtcaggaatgaagtgaaaaactaagagatgatggatgatgataggggctgtgaaaggaaaacaaatcttggggcccccaaatcactaagctaaaggagaaagtcaagctgggaactgtttagggcaatcctgcctcccattttattca

SEQ ID NO: 13 - DNA, 401 bp, Homo sapiens
tattactgctgagaaaactgggtttgataaactaaagatgcccatgtatatcagtcatgctcctggtgagaacaggtggctcactgcataatgagaggaatattcaattaactatttacaaagctatggatgacatgtagggaagccacagagagagtacagtatctagagctagtaagagtagaaggccatcactgtccacaggcctaaaggaggtagagcagtcaaaggaaacaagagacaagggaggctgcgaggacagggccacctggcagagccataaccttaaactaggtagtcacttcttggcaactctgcaggtagggagccaacctcacttttaaccctccctctgatgcccagctggtttaccccattggtgaaaatcagtgggtgaggga

SEQ ID NO: 14 - DNA, 401 bp, Homo sapiens
catgaaaagatacttaacattgtaacatctttgcattagggaactgcaaatcaaaatcataacaaaatagtactgcatgctcattaggatgactataatccaaaagaataaaaaagaaaataacaggtgttggtagggatacagatatagagaaactggagctctcatgccttgctggggggcatgtaaaatgattctgccgctttggaaaacagtttggtggttcctcaaaaagttaaacatataatccaacaattccacccaaaagaattgaaagcagggtctagtacaccaacgttcatagcagctttattcacatcaagccaaaggtggaagcagcccaaatgtctactgatggatgagttgatacacaaaatgtggtatatatatgcaatggaata

SEQ ID NO: 15 - DNA, 401 bp, Homo sapiens
gccacttgaatgccccaaaatggagagatgggcgtgggaagagaaagacacctcagcaacacagagctgagaaaacactgtgagttttatttaattcctacttaccgttattttgcatagtaaacaaaagggatatttttgaaaatccctttggataatttctgccacctaaaattctgagcattttgactcactgccttataaaaagaatcaattaattgaataagagaagggattctcccctgatcttttcaagaatccttaaaaggcacatttctcactaaggatcttgaaagtgtatttctagccaatcccaggagtcactgctcagagatttacatttcacaaatgtaatcaacagcctaagcagaatattgacgtttggactgcagagctctgct

SEQ ID NO: 16 - DNA, 400 bp, Homo sapiens
agggtccccaaatatttccatttgagatgacaaagtgctcttcagtcatttagcttactcttcagttcagatgacttatcatcttgatttcagagagttcatatatgtctgttttaaaaaactggttcaaaaagtctgaagttacgaaactaaaccaaatatgcattactctcatgtcaaattacaagctcttagctgcggggattttttcacatgcagcctggagcccttgaaaacctctgttttctgttagactctccagggtacacagaagttgcctcattattttagttaatggtgactgcaaataagccccccaagtcatttaactatgtgcttaccactgctttaaaagaaccccaagttaggtcctcatgtaggtaaaggagctcccttcaca

SEQ ID NO: 17 - DNA, 401 bp, Homo sapiens
acttgggcccaaaggcattcaactagaaagctggtaataataacagcgacagtttattgagtcttagtgtttctgagaacttttctaagtactttacacatattaaatttttaaatcttcacattagtcctgtgaggaaggtactattgttatgtctgtattacccatggggatactgacgcacaaagaagtcaagtaatatatttaagattctagtaagtgcagagcccaggtgcatgcagtgcctgggctctgccacccatgcagtgctgactagggcttccacccatggattttttttttttttttttttttttgagacagagtttcgctcttgttgcccaggctggagtgcaatggcatgatctcggctcaccacaacctccgcctctcgggttcaa

SEQ ID NO: 18 - DNA, 401 bp, Homo sapiens
cccataataatgaaggattggacctgataatctatcaggtacattttagcctgaaatttatttgtacacacgcacaaacacagacatgtgcacacacacatacacatatatatataacatttataaattttaaaacataaagctatactagaaatgaaagcttatatattgaactgccccacctttctatttgcagccagctaccaccccagtctaatgtttcactttatataaattcatttattcttttactcatttcaaatatatgatgatgtaactataaaatcaacatttagtcactctgaataacccaaaatagcaaataatttaaaaatcacttccacttgactttagaatctattacatgcattgtttttccagaaaatttacctcataattat

SEQ ID NO: 19 - DNA, 401 bp, Homo sapiens
ctacttatatgattagagaacaagaatactagggggaaaatcagcatgcatataatctaagaaattgtcattataattttaaaatcctttgcaaaatcagtaaatatgagtttaacttatataatgatacacacacacactgatatgatgctttattgtctaaacactggctgcttgtggagacgtattctggtaacaaaaaatatagcatcttaaaattgatgctagcattgtatatccaaatagagagtaaatgcaaccagaatattttttatatgtttaacattgtagtgttgctgacatcattatatatttggttatgttaatctcaaaatgcacaatatagctgtatgatctgtataatgcaaaaaaatgtagagcttcattttgatatttattat

SEQ ID NO: 20 - DNA, 401 bp, Homo sapiens
ctggaagggaacaatggaagaggtgcattagtcacattccaaaatgcaggaagcaataacatgtggcactattgtcatttatgtagcaccctaaatactgggacaaatgacatagatgcccttctgtgattactaaactcccccacagtgtctcagaaggaagagcttttgacaggaaatcatcaagatctgatgacattagagagcaattaacattctcttcaaccatgaactaattgcctcattcacatttttctagccatcctaggaagcagataataagcagcaattgtcctgcccaggaattctgacttgtgtaatttgtaaagcttttctttgtatctatttctttcctgtggccatctttttgtttttggactgtttggtaacagtaagtgggt

SEQ ID NO: 21 - DNA, 401 bp, Homo sapiens
atcattcagtattaagagagaaatgaatacattttcagatatacaagaattcagtttacctcccacagaatctctgaaaaaaatattagattactatagttaaaaaggaaaataaaataagtcctgttagaaataattggtaaaaaagcaaaggtgatgaaaacttattgaaatatattattaaagtaattgttaaaaatatacactaaatctagaatatataaatgtagcagttgttaaggggaaggggaaatagaagtggaagaaaatgaacattagaacaaatgtttagcagtgggattattttattggaagtctaatgtaagaagtatattctccagggaggtatttcaaggacatatgaatagtaaagggataataaaacaactctataaggtagt

SEQ ID NO: 22 - DNA, 401 bp, Homo sapiens
cacacacaacgctgggcccagtaaataagttttgttttttcccagggaaaagttgaacaacaatggtgagaccaggaaggctctccgttcacaggaaatactgtgtcaccgctcggccgcaggctgtgtgaggtcacgggcgacgctcgggtcacgtgtggcggctcctgttcacagtgccgtgtgtgataaactgggaccttctggtgaggggagactggcggggggtggggagggcaaggagtgggaaagtcgcctataaatgtttaacaaaagatccgcaatgggaacaggaacttgcattctttctttcaatggacaaagcttccacatcaagatacgcttgtgtgctgggaccaaatgccacagtgcggcgaaactcgtgagcacaagtcctgcgt

SEQ ID NO: 23 - DNA, 300 bp, Homo sapiens
acaacagggtatcctagcccagcaaaattgactcataaatttaatgatcacgcaattggtaattctaaatccagtcagaagtctacattctgtgtccacagtgtcatgtctagatgttggtccagtctcccatggactgtgccttgttatttgttttctctttgctaagccacatcccctgagggctctgtttatgctcactgcaaaatctttgactttttaacttactgggcatattgtcttcctacttttgttctcttctgttattttatttacttgactctgacatgtctcattccc

SEQ ID NO: 24 - DNA, 401 bp, Homo sapiens
tttcaatgggactggttggacagtgggtgcagcccatgaagggcaagccaaagcaggccggggcatcacctcacccgggaagcacaaggggtcaggcgatttctctttcctagtcaagggaagccatggcagactgcacctggaaaaacgagacacttccacccaaatactgcgtttttcgcaaggtcttagcaactaacagacaaggagattctctcccgtgcctggctcggctggtcccacacccacggtgacttgttcactgctagcacagcagtttgagatcgaactacgaggcaacaacctggctaagggaggggcatctgccattgctgaggcttgagtaggtaaacaaagtggccaggaagctcgaactgggtggagcccactgcagcttagca

SEQ ID NO: 25 - DNA, 298 bp, Homo sapiens
actttagggactttgagtgatggacaaccccctatcagatatcatcagcctgaaacatccttatcttggcattaaattagaaggaaccccagaccctgcgtaccagaattgttagaatcacagtctcagtaaagaaccaactcctgatcacttctctaaaggaaagttctagaagtctgcacactctgcagtcactttcaattctatccaagtgtacacttagaactctagaaaacactacggacagtcttcagccaggtaaagcctaaaaccagcaaagaacagggagagtgaggga

SEQ ID NO: 26 - DNA, 401 bp, Homo sapiens
cggattatcacagttctcaaaagaggagtatgcatttgcttgctccaattcctcttcttctacactctcttaagcattcctcaaccagtctaatatctcatagttccccaaaactgctctgttcaagaccattagtaagatctttgatgttaatctgtggaccgtatctctgtccttattttacttgaagcccaacagcaaataaaaaagttgttctcctctcctccctgctacactttccttatgtggcttgctgggctcctcagtcccctgtgaaaaactctgacatggagatactgcagaccagtagaagggctgggcagacactatacagaaacagtatgccctacatgctccttggctaaatctctagaatttttttcagaactcatccacaaatt

SEQ ID NO: 27 - DNA, 401 bp, Homo sapiens
attttatctcatttacttattaaatcaaaccaatattttatgaagtgattccagtattggaataaaaatgtaattctttaatcattaaaaaatctttatgaataccttacatcaactgtaggggaccaaccagggaaaagcagggagacttgtagaatctacacctccagaacaaccgacctccatcttctggacaactcatcttctaaagtgcaggacagactagttgggggagaaaggaggaaatgaaagagatagactaaaagggagggagagaacagatattttttaagtacctgttatgttctggatacagcacagagtacattgtatctattattataaggcataaagaaagatttctcaggtttttggagtcagattgcaatataaaataatag

SEQ ID NO: 28 - DNA, 401 bp, Homo sapiens
taacttcaaattgttttgcaaaatctctgtcataaaaatgcttaccaacaaatactgatactaaatttagatgtgggggtattagttataatcctgaagtgggagggggaacttcttaattccaatttagttctaagagaaggaagagtatttaggcccagagaaggttacgcttaaaggtctgatagtgttttctttgaaaaatatgtctcaaactagagaataaaactaattatctcatctaagttacctagagacatttatgctcatcagtttgataaaggactgcaagtagacacagaagctgtattttcagtcttgaacccagcaatagtacattaacaagattggggcaaggcaaagggacttttgtggcacaagatacaatatatggattgcgt

SEQ ID NO: 29 - DNA, 401 bp, Homo sapiens
ctcttcaaaggcctttgcccttgggtaccacaggttctgagacaagagggctatggagagcccccattatagctggagcctcctgccctgcccaaaggtgtgacttgaagggtggaatttcaggcagcgtggctcgccccagggaggcaaagaggccaggggaatcttcaaaggccctgggctcatcccagctaggaggcaggcacagtcataaccctaatccagtgaactcagccctcatcctgactctcatggtattctgtcccagggagcctctttccagctttcttagaagctttaatgtcagcacttgcagggccttagaaactgcacgctacctcttcatttcatacatgaggaaactgaggcccagggtggacacagggctgcccagcgagtta

SEQ ID NO: 30 - DNA, 401 bp, Homo sapiens
attataaagcaaagcactaacctcatagaatacctgagtcaagttccctgtgttctcattttctagcctcttctaccagacactatgaaaaataacagccccatctctccagaaaatcttaggagatataggcgtgctgaatttaaggtgtctgtggcacatgcaagtggatcagccactgggctgtccagaatgcaagacagaactcagagttggggacataaacttggcagtcatctgtgtaaaagagaaaaggtaggtaaagtcccacaaggatgggttggcctacagaaggcacagagagaagagagcctggtttagcatgggctacgatcagagtccctgggttcaaatcttggctccacccatttctatctggttgctctagggcatgttaccta